(19) United States
(12) Patent Application Publication
(10) Pub. No.: US 2005/0032045 A1
(43) Pub. Date: Feb. 10, 2005

(54) Chimeric adenovirus capsid proteins

(76) Inventors: Suresh K. Tikoo, Saskatoon (CA); Alexandre N. Zakhartchouk, Saskatoon (CA); Lorne A. Babiuk, Saskatoon (CA); Radhey S. Kaushik, Brookings, SD (US); Philip J. Griebel, Saskatoon (CA)

Correspondence Address: MORRISON & FOERSTER LLP, 755 PAGE MILL RD, PALO ALTO, CA, 94304-1018, US

(21) Appl. No.: 10/866,013

(22) Filed: Jun. 10, 2004

Related U.S. Application Data
(60) Provisional application No. 60/477,539, filed on Jun. 10, 2003.

(51) Int. Cl. C12Q 1/70; C07K 14/075

(52) U.S. Cl. 435/5; 530/350

(57) ABSTRACT

The present invention relates to chimeric adenovirus capsid proteins comprising a part of or all of an adenovirus capsid protein and a binding partner of a cell-surface binding site on a cell present in gut associated lymphoid tissues (GALT) of a mammal, wherein the chimeric adenovirus capsid protein is capable of binding the cell present in gut associated lymphoid tissues (GALT). In some examples, the adenovirus capsid protein is located on the surface of the adenovirus capsid. The present invention provides adenovirus capsids comprising a chimeric capsid protein. The present invention also provides complexes comprising a chimeric adenovirus capsid protein bound to a cell in GALT. The present invention also provides encapsidation systems and vectors, in particular adenovirus vectors, capable of expressing a chimeric adenovirus capsid protein encompassed within the invention. The present invention also provides viral particles, host cells and compositions comprising such vectors, in particular for use in vaccines, methods of eliciting an immune response and methods for targeted delivery of heterologous proteins, such as antigens, to target cells.

### CROSS REFERENCE TO RELATED APPLICATION

This application claims benefit of U.S. Provisional Patent Application Ser. No. 60/477,539, filed Jun. 10, 2003, which is incorporated by reference herein in its entirety.

### TECHNICAL FIELD

This invention relates to chimeric adenovirus capsid proteins and encapsidation systems comprising vectors expressing the chimeric adenovirus capsid proteins, in particular for targeted delivery. The invention also relates to methods of making and using the chimeric adenovirus capsid proteins and vectors expressing them.

### BACKGROUND OF THE INVENTION

The adenoviruses cause enteric or respiratory infection in humans as well as in domestic and laboratory animals. For a general review of Adenoviridae, see Fields et al. Fundamental Virology (1991, 2nd edition, Raven Press, New York, chapter 31). For general background references regarding adenovirus and development of adenoviral vector systems, see Graham et al. (1973) Virology 52:456-467; Takiff et al. (1981) Lancet 11:832-834; Berkner et al. (1983) Nucleic Acid Research 11: 6003-6020; Graham (1984) EMBO J. 3:2917-2922; Bett et al. (1993) J. Virology 67:5911-5921; and Bett et al. (1994) Proc. Natl. Acad. Sci. USA 91:8802-8806.

BAVs are common pathogens of cattle usually resulting in subclinical infection (Darbyshire et al., 1965. J. Comp. Pathol. 75:327-330), though occasionally associated with a more serious respiratory tract infection (Darbyshire et al., 1966 Res. Vet. Sci. 7:81-93; and Mattson et al., 1988 J. Vet Res 49:67-69). Porcine adenovirus (PAV) infection has been associated with encephalitis, pneumonia, kidney lesions and diarrhea (Derbyshire, 1992 In: “Diseases of Swine” (ed. Leman et al.), 7th edition, Iowa State University Press, Ames, IA. pp. 225-227). It has been shown that PAV is capable of stimulating both humoral response and a mucosal antibody responses in the intestine of infected piglets. Tuboly et al. (1993) Res. in Vet. Sci. 54:345-350. Human adenovirus sequences are described in for example, Foy H. M. (1989) Adenoviruses In Evans AS (ed). Viral Infections of Humans. New York, Plenum Publishing, pp 77-89 and Rubin B. A. (1993) Clinicalpicture and epidemiology of adenovirus infections, Acta Microbiol. Hung 40:303-323.

Adenoviruses generally undergo a lytic replication cycle following infection of a host cell. In addition to lysing the infected cell, the replicative process of adenovirus blocks the transport and translation host cell mRNA, thus inhibiting cellular protein synthesis. For a review of adenoviruses and adenovirus replication, see Shenk, T. and Horwitz, M. S. (Virology, third edition, Fields, B. N. et al., eds., Raven Press Limited, New York (1996), Chapters 67 and 68, respectively).

The application of genetic engineering has resulted in several attempts to prepare adenovirus expression systems for obtaining vaccines. U.S. Pat. Nos. 6,001,591, 5,820,868 and 6,319,716 disclose recombinant protein production in bovine adenovirus expression vector systems. U.S. Pat. No. 6,492,343 discloses recombinant protein production in porcine adenovirus expression vector systems. U.S. Pat. No. 5,922,576 discloses systems for generating recombinant adenoviruses.

Krasnykh et al. (1996, Journal of Virology, 70:6839), and Zabner et al. (1999, Journal of Virology, 73:8689) report generation of human adenovirus vectors with modified fiber regions. Xu et al. (1998, Virology, 248:156-163) disclose an ovine adenovirus carrying the fiber protein cell binding domain of human Adenovirus Type 5. Adenovirus capsid protein IX (pIX), a 14.3 kDa minor structural component of human adenoviruses, is disclosed in PCT publications WO 02/096939 and WO 01/58940; Colby et al., (1981, J. Virol. 39:977-980); Akalu et al., 1999, J. Virology, 1999, 73:6182-6187; and Dmitriev et al., 2002.

All references and patent publications are hereby incorporated herein in their entirety.

### BRIEF SUMMARY OF THE INVENTION

The present invention provides chimeric adenovirus capsid proteins wherein said proteins comprise a part of or all of an adenovirus capsid protein and a binding partner of a cell-surface binding site present in a cell of gut associated lymphoid tissue (GALT) of a mammal, wherein said chimeric adenovirus capsid protein is capable of binding to the cell. In some examples, the adenovirus capsid proteins are selected from the group consisting of hexon, penton, fiber, pIX, IIIa, VI and VIII proteins. In other examples, the adenovirus is mammalian adenovirus, such as a human, porcine, bovine, or ovine adenovirus. In some examples, the mammalian adenovirus is a ruminant adenovirus. In other examples, the binding partner is an antibody, such as a monoclonal antibody, or a fragment thereof. In some examples, the antibody is a single chain antibody. In yet other examples, the antibody specifically binds an epithelial cell present in the GALT. In additional examples, the antibody binds a cell present in the Peyer's patches. In further examples, the antibody binds a microfold (M) cell. In further examples, the antibody binds a cell-surface binding site present in a cell in mammalian GALT and cross reacts with heterologous mammalian species. In some examples, the antibody binds a protein present on the surface of the cell and in yet other examples, binds a carbohydrate present on the surface of the cell.

In some examples, a chimeric adenovirus capsid protein is encoded by a polynucleotide comprising nucleic acid encoding a part of or all of said capsid protein and nucleic acid encoding an amino acid sequence for said binding partner. In other examples, a chimeric adenovirus capsid protein comprises a part of or all of the capsid protein conjugated to the binding partner. The present invention also provides encapsidation systems capable of expressing an adenovirus capsid comprising a chimeric adenovirus capsid protein as well as complexes comprising a chimeric adenovirus capsid protein bound to a cell-surface binding site present in a cell of GALT.

The present invention also provides recombinant vectors encoding a chimeric adenovirus capsid protein including replication-competent and replication-defective adenovirus vectors. In some examples, the adenovirus vector is a mammalian adenovirus vector, including human, porcine, bovine and sheep adenovirus. In further examples, the mammalian adenovirus vector is a ruminant adenovirus vector. In additional examples, a vector further comprises adenovirus sequences essential for encapsidation, including bovine, porcine and human adenovirus sequences. In some examples, a replication-deficient bovine adenovirus vector lacks E1 function and in additional embodiments comprises a deletion of part or all of the E1 gene region and/or a deletion of part or all of the E3 gene region. In yet additional examples, a vector further comprises a polynucleotide encoding a heterologous protein, such as for example, an antigen of a mammalian pathogen, including bovine, porcine, ovine, human, feline, and canine pathogens. The present invention also encompasses compositions, such as immunogenic compositions and vaccine compositions, host cells and viral particles comprising a chimeric adenovirus capsid protein or a vector that expresses a chimeric adenovirus capsid protein. In some examples, compositions further comprise a pharmaceutically acceptable excipient.

The present invention also provides methods for eliciting an immune response in a mammalian host comprising administering an immunogenic composition comprising a vector or viral particles that express a chimeric adenovirus capsid protein to the mammalian host. In some embodiments, the immunogenic composition is administered orally. In other embodiments, the mammalian host cell is a ruminant mammal, such as a bovine or ovine mammal.

The present invention also provides methods for producing a chimeric adenovirus capsid protein comprising conjugating a binding partner of a cell-surface binding site of a cell present in the GALT to part of or all of an adenovirus capsid protein wherein said capsid protein is located on the surface of the adenovirus capsid. The present invention also provides methods for preparing a recombinant adenovirus comprising a polynucleotide encoding a chimeric adenovirus capsid protein comprising the steps of culturing a suitable host cell transformed with or comprising an adenovirus vector capable of expressing a chimeric adenovirus vector under conditions suitable to allow formation of a virus particle from said vector and optionally recovering the virus.

### BRIEF DESCRIPTION OF THE DRAWINGS

FIGS. 1A-1C. Maps of the plasmids which have been used for construction of BAV-3 recombinants.

FIG. 2. Sequence of the BAV-3 pIX-YFP chimerical gene and fusion protein.

FIG. 3. Sequence of the BAV-3 pIX-RGD chimerical gene and fusion protein.

FIG. 4. PCR analysis of the viral DNA. 1-wild-type BAV-3; 2-BAV950, passage 2; 3-BAV950, passage 10; 4-BAV951, passage 2; 5-BAV951, passage 10.

FIGS. 5A-5B. Western blotting analysis to detect BAV-3 pIX in the purified virions. (5A) Lane 1-BAV-3; lane 2-BAV950. (5B) Lane 1-BAV-3; lane 2-BAV951.

FIGS. 6A-6B. Immunoelectron microscopy of the purified virions. (A) BAV-3. (B) BAV951.

FIG. 7. Number of the viral genomes in the infected cells, estimated by Real Time PCR.

### DETAILED DESCRIPTION OF THE INVENTION

The present invention relates to chimeric adenovirus capsid proteins, adenovirus vectors comprising nucleic acid encoding chimeric adenovirus capsid proteins, and encapsidation systems expressing adenovirus capsids (that is, chimeric adenovirus capsids) that comprise a binding partner for a cell-surface binding site on a cell present in gut associated lymphoid tissues (GALT). In some examples, the adenovirus vectors are used, in particular, for targeted delivery of a protein or polypeptide to the GALT of a mammal. In some examples, such adenovirus vectors, encapsidation systems and adenovirus capsids are used to target delivery of an antigen, such as an antigen of a mammalian pathogen, to the GALT for the purpose of inducing a mucosal immune response to the antigen. In some examples, the binding partner is an antibody, such as a monoclonal antibody, or a fragment thereof, or a minimal recognition unit thereof. In an illustrative example, the binding partner for a cell-surface binding site on a cell present in GALT is a monoclonal antibody that is cross reactive with (that is, that specifically binds) bovine, porcine and ovine jejunum Peyer's patches (PP). Such an antibody provides the advantage of having one binding partner that cross reacts with a cell in GALT of several mammalian species. In other examples, the binding partner specifically binds a cell-surface binding site on an GALT microfold (M) cell. The use of adenovirus vectors in oral vaccines for farm animals, especially ruminant mammals such as cows and sheep, has been problematic due to the presence of chambered stomachs and the degradation of the vector in the digestive tract. The present invention provides adenovirus vectors, including adenovirus vectors comprising nucleic acid encoding chimeric adenovirus capsid proteins that comprise a binding partner for a cell-surface binding site on a cell present in GALT, and encapsidation systems that produce such adenovirus capsids that are degraded to a lesser extent in animal models than comparable adenovirus capsids or adenovirus vectors lacking the binding partner, or lacking nucleic acid encoding the binding partner.

Accordingly, the present invention provides chimeric adenovirus capsid proteins, wherein said chimeric adenovirus capsid proteins comprise a part of or all of an adenovirus capsid protein and a binding partner of a cell-surface binding site on a cell present in gut associated lymphoid tissues (GALT) of a mammal, wherein the chimeric adenovirus capsid protein is capable of binding the cell (the cell-surface binding site) present in gut associated lymphoid tissues (GALT). In some examples the adenovirus capsid protein is located on the surface of the adenovirus capsid. Vectors, particularly adenovirus vectors, comprising chimeric adenovirus capsid proteins of the present invention that bind a cell present in the GALT and which express heterologous proteins, such as an antigen of a mammalian pathogen, are particularly advantageous for use as oral vaccines for mammals. In some examples, the chimeric adenovirus capsid protein is homologous to the cell present in GALT (such as, for example, a bovine adenovirus capsid protein for use in immunizing bovine mammals) and in other examples, the chimeric adenovirus capsid protein is heterologous to the cell present in GALT (such as the use of a bovine adenovirus capsid protein for use in immunizing non-bovine mammals).

In some examples, the binding partner is an antibody, such as for example, a monoclonal antibody, or fragment thereof, that specifically binds a protein or other structure, such as for example, a carbohydrate, on a cell present in GALT. In some examples, the binding partner specifically binds a cell present in the epithelium of GALT. In other examples, the binding partner specifically binds microfold (M) cells of GALT. In some examples, the present invention provides chimeric adenovirus capsid proteins encoded by a polynucleotide comprising nucleic acid encoding a part of or all of at least one capsid protein and said binding partner. In other examples, the chimeric adenovirus capsid protein comprises a part of or all of the an adenovirus capsid protein covalently bound or conjugated or linked to the binding partner. Accordingly, the present invention provides adenovirus capsids comprising a chimeric capsid protein (and adenovirus vectors encoding them). The present invention also provides complexes comprising a chimeric adenovirus capsid protein bound to a cell in GALT of a mammal and compositions comprising such complexes.

The present invention also provides vectors, such as viral vectors including but not limited to adenovirus vectors, comprising polynucleotides encoding the chimeric adenovirus capsid protein and at least one adenovirus encapsidation sequence, wherein said vectors are capable of forming adenovirus capsids. The present invention provides viral vectors, such as replication-competent and replication-deficient adenovirus vectors, comprising polynucleotides encoding a chimeric adenovirus capsid protein. In some examples, the adenovirus vectors lack one or more nucleic acids encoding adenovirus proteins essential for replication and are replication-deficient. The invention also relates to host cells and viral particles comprising chimeric adenovirus capsid proteins of the present invention as well as methods of making and using the chimeric adenovirus capsid proteins and vectors of the present invention in particular for use in vaccine compositions, methods for eliciting an immune response, and methods of delivering a nucleic acid encoding a protein, such as for example, an antigen of a pathogen, to a target cell.

In some illustrative examples, the present invention provides a chimeric adenovirus capsid protein, and vectors expressing the protein, wherein the protein comprises a part of or all of an adenovirus capsid pIX. In some examples, the binding partner is a fragment or a minimal recognition unit of an antibody that binds a cell present in GALT of a mammal. In other examples, the present invention provides production of single chain antibodies to a cell-surface binding site present in a cell present in GALT of a mammal. In some examples, the binding partner of a cell (that is, a cell-surface binding site), in particular, is a single chain antibody to a cell present in GALT, such as for example, an M cell.

General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000);
A “chimeric adenovirus capsid protein” as used herein means that the protein comprises a part of or all of at least one adenovirus capsid protein and a binding partner for a cell-surface binding site of a cell present in gut associated lymphoid tissue (GALT). In some examples the adenovirus capsid protein is located on the surface of the adenovirus capsid, such that the binding partner is displayed on the surface of the adenovirus capsid and is available for binding. The present invention encompasses chimeric adenovirus capsid proteins wherein the binding partner of the chimeric capsid protein is present within or at the N-terminus of the adenovirus capsid protein, present within or at the C-terminus of the adenovirus capsid protein, or present internal to the adenovirus capsid protein (as long as the binding partner is capable of being displayed on the surface of the capsid). The present invention encompasses a chimeric adenovirus capsid protein comprising a part of an adenovirus capsid protein located on the surface of the capsid, as long as an adenovirus vector comprising nucleic acid encoding the part of the chimeric adenovirus capsid protein is capable of forming an adenovirus capsid. In some examples, the binding partner of the chimeric capsid protein is fused to a part of or all of an adenovirus capsid protein, that is the chimeric adenovirus capsid protein is encoded by a polynucleotide comprising nucleic acid encoding a part of or all of the adenovirus capsid protein and nucleic acid encoding an amino acid sequence for the binding partner with or without nucleic acid sequences in between. In other examples, the binding partner of the chimeric capsid protein is covalently bound or conjugated or linked to the adenovirus capsid by any means known in the art. The binding partner may be bound, linked or conjugated directly or indirectly through a spacer group. Adenovirus capsid proteins are known in the art and are disclosed herein. As disclosed in Fields Virology, Third Edition (ed. Fields et al., pub. Lippincott-Raven, page 2115) adenovirus capsid proteins include hexon, penton, fiber proteins and proteins IIIa, VI, VIII, and IX. The present invention encompasses the use of any adenovirus capsid protein as long as the protein is located on the surface of the adenovirus capsid or is capable of displaying a binding domain for a cell-surface binding site on a cell in gut-associated lymphoid tissue (GALT) on the surface of the capsid. In some examples, a chimeric adenovirus capsid protein encompasses at least one of the following adenovirus capsid proteins located on the surface of the capsid: hexon, penton, fiber, pIX, and IIIa. In some examples, a chimeric adenovirus capsid protein comprises a part of or all of adenovirus capsid protein IX. In other examples, a chimeric adenovirus capsid protein comprises a part of or all of adenovirus capsid fiber protein. An “encapsidation system” as used herein refers to a system comprising a vector and optionally a helper cell that comprises adenovirus sequences necessary to form an adenovirus capsid and may or may not comprise viral proteins. For example, viral proteins essential for adenovirus replication, or adenovirus encapsidation for example, may be provided by a helper cell.

A “binding partner” as used herein refers to any suitable molecule or entity that is capable of binding a cell-surface binding site on a cell present in GALT tissue and includes, but is not limited to cell-binding proteins, including lectins, or peptides (including modified proteins and peptides, such as for example, glycoproteins and mucoproteins); amino acids motifs, or short stretches of amino acids, such as those constituting a peptide hormone; domains of polypeptides that can fold independently into a structure that can bind a target cell; carbohydrates, including mono-, di- and oligosaccharides; lipids; mucin molecules; antibodies, including but not limited to monoclonal antibody, or a fragment thereof capable of binding to a cell-surface binding site, a single chain of an antibody, a single domain antibody or a minimal recognition unit of an antibody. The binding partner may be a part of an antibody (for example a Fab fragment) or a synthetic antibody fragment (for example, ScFv).

Gut associated lymphoid tissue (GALT), a major component of the immune system, is a term well understood by one skilled in the art and includes but is not limited to cells of the Peyer's patches (PP), aggregates of subepithelial lymphoid follicles located in the mucosa of the small intestine, which may be located in the ileal PP, jejunal PP, and other jejunum; the specialized microfold (M) cells of the PP, the overlying epithelium devoid of villi; intestinal epithelial cells, including follicle associated epithelium of PP, and enterocytes. In the small and large intestine, M cells are present in the dome areas of the Peyer's patches and other GALT tissues. See Gebert et al. (1996, Int. Rev. Cytol. 167:91-151), specifically incorporated by reference herein in its entirety for disclosure on GALT.

As used herein, “region(s) essential for encapsidation”, an “encapsidation region”, “sequence(s) essential for encapsidation”, an “encapsidation sequence” and a “packaging domain” or “packaging motif” (used interchangeably herein) refer to the sequence(s) of an adenovirus genome that is/are necessary for inserting the adenovirus DNA into adenovirus capsids. In some examples, an encapsidation sequence is cis-acting. The present invention encompasses the use of any mammalian sequence essential for encapsidation, such as for example, bovine, ovine, porcine, and human as long as the sequence is capable of inserting the adenovirus DNA into adenovirus capsids. A “bovine adenovirus” sequence essential for encapsidation encompasses any bovine adenovirus sequence essential for encapsidation as long as the sequence is capable of inserting the adenovirus DNA into adenovirus capsids. Illustrative examples are disclosed herein. In some examples, a bovine adenovirus sequence essential for encapsidation is a BAV-3 sequence. A “bovine adenovirus sequence(s) essential for encapsidation that is heterologous to the adenovirus vector”, means that the adenovirus vector sequence is a non-bovine adenovirus sequences or the adenovirus vector sequence is a bovine adenovirus sequence of a different serotype than the bovine adenovirus sequence essential for encapsidation. The heterologous adenovirus vector sequences are not limited and can be any adenovirus sequence as long as the bovine adenovirus sequence(s) essential for encapsidation can function to insert the adenovirus DNA into an adenovirus capsid. In some examples, a bovine adenoviral sequence(s) essential for encapsidation is used in an adenovirus vector that comprises bovine adenovirus sequences. All BAV3 nucleotide numbering herein is with respect to the left end of the adenovirus and the BAV3 reference sequence provided in GenBank accession number AF030154. A “porcine adenovirus” sequence(s) essential for encapsidation encompasses any porcine adenovirus sequence(s) essential for encapsidation as long as the sequence is capable of inserting the adenovirus DNA into adenovirus capsids. Illustrative examples are disclosed herein. As used herein, the phrase, “porcine adenovirus sequence(s) essential for encapsidation that is heterologous to the adenovirus vector”, means that the adenovirus vector sequences are non-porcine adenovirus sequences or are of a different serotype than the porcine adenovirus sequence essential for encapsidation. The heterologous adenovirus vector sequences are not limited and can be any adenovirus sequence as long as the porcine adenovirus sequence(s) essential for encapsidation can function to insert the adenovirus DNA into an adenovirus capsid. In some examples, a porcine adenoviral sequence(s) essential for encapsidation is used in an adenovirus vector that comprises porcine adenovirus sequences. An adenovirus vector may be constructed to comprise multiple adenovirus sequences essential for encapsidation, for example, multiple identical sequences or multiple different sequences, or the adenovirus vector encapsidation sequence may be heterologous to the adenovirus vector. Human adenovirus encapsidation sequences are known in the art. See for example, Grable et al. 1990, J. Virol. 64:2047.

An “adenovirus vector” or “adenoviral vector” (used interchangeably) comprises a polynucleotide construct of the invention. A polynucleotide construct of this invention may be in any of several forms, including, but not limited to, DNA, DNA encapsulated in an adenovirus coat, DNA packaged in another viral or viral-like form (such as herpes simplex, and AAV), DNA encapsulated in liposomes, DNA complexed with polylysine, complexed with synthetic polycationic molecules, conjugated with transferrin, and complexed with compounds such as PEG to immunologically “mask” the molecule and/or increase half-life, and conjugated to a nonviral protein. Preferably, the polynucleotide is DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides. Adenovirus vectors may be replication-competent or replication-deficient in a target cell. Replication-deficient adenovirus vectors can be propagated in appropriate helper cell lines expressing adenoviral proteins essential for replication that are lacking from the replication-deficient adenovirus. “Replication-deficient” and “replication-defective” are used interchangeably herein.

As used herein, the term “altered tropism” refers to changing the specificity of an adenovirus. The term “altered tropism” encompasses changing species specificity as well as changing tissue or cell specificity of an adenovirus. The present invention encompasses chimeric adenovirus capsid proteins, vectors, adenovirus vectors and viral particles that exhibit cell specificity, such as for example, cell specificity for a cell present in GALT, and may additionally exhibit altered species tropism. In some examples the chimeric adenovirus capsid proteins, vectors, adenovirus vectors and viral particles are heterologous to the species of the target cell in GALT.

An “antibody” is an immunoglobulin molecule capable of specific binding to a target, such as, for example, a protein, peptide, carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)₂, Fv), single chain (ScFv), mutants thereof, fusion proteins comprising an antibody portion, humanized antibodies, chimeric antibodies, and any other modified configuration of the immunoglobulin molecule that comprises an antigen recognition site of the required specificity. An antibody includes an antibody of any class, such as IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known

A “monoclonal antibody” refers to a homogeneous antibody population wherein the monoclonal antibody is comprised of amino acids (naturally occurring and non-naturally occurring) that are involved in the selective binding of an antigen. Monoclonal antibodies are highly specific, being directed against a single antigenic site. The term “monoclonal antibody” encompasses not only intact monoclonal antibodies and full-length monoclonal antibodies, but also fragments thereof (such as Fab, Fab′, F(ab′)₂, Fv), single chain (ScFv), mutants thereof, fusion proteins comprising an antibody portion, humanized monoclonal antibodies, chimeric monoclonal antibodies, and any other modified configuration of the immunoglobulin molecule that comprises an antigen recognition site of the required specificity and the ability to bind to an antigen. It is not intended to be limited as regards to the source of the antibody or the manner in which it is made (e.g., by hybridoma, phage selection, recombinant expression, transgenic animals, etc.).

An epitope that “specifically binds” or “preferentially binds” (used interchangeably herein) to an antibody or a polypeptide is a term well understood in the art, and methods to determine such specific or preferential binding are also well known in the art. The term is also used interchangeably with “antigenic determinant” or “antigenic determinant site.” A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a microfold (M) cell epitope is an antibody that binds this M cell epitope with greater affinity, avidity, more readily, and/or with greater duration than it binds to other epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. A “variable region” of an antibody refers to the variable region of the antibody light chain or the variable region of the antibody heavy chain, either alone or in combination. A “constant region” of an antibody refers to the constant region of the antibody light chain or the constant region of the antibody heavy chain, either alone or in combination.

An “antigen” refers to a molecule containing one or more epitopes that will stimulate a host's immune system to make a humoral and/or cellular antigen-specific response. The term is also used interchangeably with “immunogen.”

The terms “polypeptide”, “oligopeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon an antibody, the polypeptides can occur as single chains or associated chains.

As used herein, the term “vector” refers to a polynucleotide construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, or a “viral vector” which is designed to result in the production of a recombinant virus or virus-like particle, or “shuttle vectors”, which comprise the attributes of more than one type of vector.

By “live virus” is meant, in contradistinction to “killed” virus, a virus which is capable of producing identical progeny in tissue culture and inoculated animals.

A “helper-free” virus vector is a vector that does not require a second virus or a cell line to supply something defective in the vector. A “helper-dependent” virus vector requires a second virus or a cell line to supply something defective in the vector.

A “double-stranded DNA molecule” refers to the polymeric form of deoxyribonucleotides (adenine, guanine, thymine, or cytosine) in its normal, double-stranded helix. This term refers only to the primary and secondary structure of the molecule, and does not limit it to any particular tertiary forms. Thus, this term includes double-stranded DNA found, inter alia, in linear DNA molecules (e.g., restriction fragments of DNA from viruses, plasmids, and chromosomes). In discussing the structure of particular double-stranded DNA molecules, sequences may be described herein according to the normal convention of giving only the sequence in the 5′ to 3′ direction along the non-transcribed strand of DNA (i.e., the strand having the sequence homologous to the mRNA).

A DNA “coding sequence” is a DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, procaryotic sequences, cDNA from eucaryotic mRNA, genomic DNA sequences from eucaryotic (e.g., mammalian) DNA, viral DNA, and even synthetic DNA sequences. A polyadenylation signal and transcription termination sequence will usually be located 3′ to the coding sequence.

A “transcriptional promoter sequence” is a DNA regulatory region capable of binding RNA polymerase in a cell and initiating transcription of a downstream (3′ direction) coding sequence. For purposes of defining the present invention, the promoter sequence is bound at the 3′ terminus by the translation start codon (ATG) of a coding sequence and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eucaryotic promoters will often, but not always, contain “TATA” boxes and “CAAT” boxes. Procaryotic promoters contain Shine-Dalgamo sequences in addition to the −10 and −35 consensus sequences.

DNA “control sequences” refer collectively to promoter sequences, ribosome binding sites, splicing signals, polyadenylation signals, transcription termination sequences, upstream regulatory domains, enhancers, translational termination sequences and the like, which collectively provide for the transcription and translation of a coding sequence in a host cell.

A coding sequence or sequence encoding a protein is “operably linked to” or “under the control of” control sequences in a cell when RNA polymerase will bind the promoter sequence and transcribe the coding sequence into mRNA, which is then translated into the polypeptide encoded by the coding sequence.

A “host cell” is a cell which has been transformed, or is capable of transformation, by an exogenous DNA sequence.

A cell has been “transformed” by exogenous DNA when such exogenous DNA has been introduced inside the cell membrane. Exogenous DNA may or may not be integrated (covalently linked) to chromosomal DNA making up the genome of the cell. In procaryotes and yeasts, for example, the exogenous DNA may be maintained on an episomal element, such as a plasmid. A stably transformed cell is one in which the exogenous DNA has become integrated into the chromosome so that it is inherited by daughter cells through chromosome replication. For mammalian cells, this stability is demonstrated by the ability of the cell to establish cell lines or clones comprised of a population of daughter cell containing the exogenous DNA.

A “clone” is a population of daughter cells derived from a single cell or common ancestor. A “cell line” is a clone of a primary cell that is capable of stable growth in vitro for many generations.

A “heterologous” region of a DNA construct is an identifiable segment of DNA within or attached to another DNA molecule that is not found in association with the other molecule in nature. Thus, when the heterologous region encodes a viral gene, the gene will usually be flanked by DNA that does not flank the viral gene in the genome of the source virus or virus-infected cells. Another example of the heterologous coding sequence is a construct wherein the coding sequence itself is not found in nature (e.g., synthetic sequences having codons different from the native gene). Allelic variation or naturally occurring mutational events do not give rise to a heterologous region of DNA, as used herein. As used herein in describing adenovirus vectors, “heterologous mammalian capsid region” or “heterologous mammalian capsid protein” means that the capsid protein is obtainable from a different mammalian species than the adenovirus vector species or is obtainable from the same species mammal but from a different type or sub-type adenovirus. For example “heterologous mammalian capsid protein” encompasses replacement of one sub-type mammalian adenovirus capsid protein with another sub-type mammalian adenovirus capsid protein as well as replacement of a mammalian adenovirus capsid protein with another species mammalian capsid protein.

“Native” proteins or polypeptides refer to proteins or polypeptides recovered from adenovirus or adenovirus-infected cells. Thus, the term “native adenovirus polypeptide” would include naturally occurring adenovirus proteins and fragments thereof. “Non-native” polypeptides refer to polypeptides that have been produced by recombinant DNA methods or by direct synthesis. “Recombinant” polypeptides refers to polypeptides produced by recombinant DNA techniques; i.e., produced from cells transformed by an exogenous DNA construct encoding the desired polypeptide.

An “immunological response” to a composition or vaccine is the development in the host of a cellular and/or antibody-mediated immune response to the composition or vaccine of interest. Usually, such a response consists of the subject producing antibodies, B cells, helper T cells, suppressor T cells, and/or cytotoxic T cells directed specifically to an antigen or antigens included in the composition or vaccine of interest.

The terms “immunogenic polypeptide” and “immunogenic amino acid sequence” and “immunogen” refer to a polypeptide or amino acid sequence, respectively, which elicit antibodies that neutralize viral infectivity, and/or mediate antibody-complement or antibody-dependent cell cytotoxicity to provide protection of an immunized host. An “immunogenic polypeptide” as used herein, includes the full length (or near full length) sequence of the desired protein or an immunogenic fragment thereof.

By “immunogenic fragment” is meant a fragment of a polypeptide which includes one or more epitopes and elicits antibodies that neutralize viral infectivity, and/or mediates antibody-complement or antibody-dependent cell cytotoxicity to provide protection of an immunized host. Such fragments will usually be at least about 5 amino acids in length, and preferably at least about 10 to 15 amino acids in length. There is no critical upper limit to the length of the fragment, which could comprise nearly the full length of the protein sequence, or even a fusion protein comprising fragments of two or more of the antigens. The term “treatment” as used herein refers to treatment of a mammal, such as bovine, ovine or human or other mammal, either (i) the prevention of infection or reinfection (prophylaxis), or (ii) the amelioration, reduction or elimination of symptoms of an infection. In some examples, the vaccine comprises a recombinant adenovirus that produces a chimeric adenovirus capsid protein.

By “infectious” is meant having the capacity to deliver the viral genome into cells.

The terms “polynucleotide” and “nucleic acid”, used interchangeably herein, refer to a polymeric form of nucleotides of any length, either ribonucleotides or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be a oligodeoxynucleoside phosphoramidate (P—NH2) or a mixed phosphoramidate-phosphodiester oligomer. Peyrottes et al. (1996) Nucleic Acids Res. 24: 1841-8; Chaturvedi et al. (1996) Nucleic Acids Res. 24: 2318-23; Schultz et al. (1996) Nucleic Acids Res. 24: 2966-73. A phosphorothioate linkage can be used in place of a phosphodiester linkage. Braun et al. (1988) J. Immunol. 141: 2084-9; Latimer et al. (1995) Molec. Immunol. 32: 1057-1064. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer. Reference to a polynucleotide sequence (such as referring to a SEQ ID NO) also includes the complement sequence.

The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracyl, other sugars and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support. Preferably, the polynucleotide is DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

A polynucleotide or polynucleotide region has a certain percentage (for example, 80%, 85%, 90%, or 95%) of “sequence identity” to another sequence means that, when aligned, that percentage of bases are the same in comparing the two sequences. This alignment and the percent homology or sequence identity can be determined using software programs known in the art, for example those described in Current Protocols in Molecular Biology (F. M. Ausubel et al., eds., 1987) Supplement 30, section 7.7.18, Table 7.7.1. A preferred alignment program is ALIGN Plus (Scientific and Educational Software, Pennsylvania), preferably using default parameters, which are as follows: mismatch=2; open gap=0; extend gap=2.

Under “transcriptional control” is a term well understood in the art and indicates that transcription of a polynucleotide sequence, usually a DNA sequence, can in some examples depend on its being operably (operatively) linked to an element which contributes to the initiation of, or promotes, transcription and in other examples, can act from a distance away, such as the case with enhancers. Bovine and porcine adenovirus E1 transcriptional control regions described herein appear to act as enhancers and do not need to be operably linked to a promoter (or other control element) and can work at a distance from the promoter (or other control element) of the gene of interest. “Operably linked” refers to a juxtaposition wherein the elements are in an arrangement allowing them to function.

In the context of adenovirus, a “heterologous polynucleotide” or “heterologous gene” or “heterologous transgene” is any polynucleotide or gene that is not present in wild-type adenovirus. Preferably, the transgene will also not be expressed or present in the target cell prior to introduction by the adenovirus vector.

In the context of adenovirus, a “heterologous” promoter or enhancer is one which is not associated with or derived from an adenovirus gene.

In the context of adenovirus, an “endogenous” promoter, enhancer, or control region is native to or derived from adenovirus.

“Replication” and “propagation” are used interchangeably and refer to the ability of an adenovirus vector of the invention to reproduce or proliferate. These terms are well understood in the art. For purposes of this invention, replication involves production of adenovirus proteins and is generally directed to reproduction of adenovirus. Replication can be measured using assays standard in the art and described herein, such as a burst assay or plaque assay. “Replication” and “propagation” include any activity directly or indirectly involved in the process of virus manufacture, including, but not limited to, viral gene expression; production of viral proteins, nucleic acids or other components; packaging of viral components into complete viruses; and cell lysis.

A polynucleotide sequence that is “depicted in” a SEQ ID NO means that the sequence is present as an identical contiguous sequence in the SEQ ID NO. The term encompasses portions, or regions of the SEQ ID NO as well as the entire sequence contained within the SEQ ID NO.

A “host cell” includes an individual cell or cell culture which can be or has been a recipient of an adenoviral vector(s) of this invention. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with an adenoviral vector of this invention.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom, and the progeny thereof. The definition also includes samples that have been manipulated in any way after their procurement, such as by treatment with reagents, solubilization, or enrichment for certain components, such as proteins or polynucleotides. The term “biological sample” encompasses a clinical sample, and also includes cells in culture, cell supernatants, cell lysates, serum, plasma, biological fluid, and tissue samples.

An “individual” or “mammalian subject” is a vertebrate, including humans, farm animals, such as cows, sheep and pigs, sport animals, rodents, primates, and pets. Ruminant mammals are known by those of skill in the art and refer to a mammal of or relating to the suborder Ruminantia that include even-toed hoofed mammals that have a 3 or 4 chambered stomach, such as for example cows, sheep, deer, giraffe and camels.

An “effective amount” is an amount sufficient to effect beneficial or desired results, including clinical results, such as decreasing one or more symptoms resulting from the disease; increasing quality of life of those suffering from the disease; decreasing the dose of other medications required to treat the disease; enhancing the effect of another therapeutic composition such as through targeting; delaying the progression of disease, and/or prolonging survival of the individual subject to the disease; and/or for veterinary use, increasing weight gain of the animal; preventing weight loss of the animal. An effective amount can be administered in one or more administrations. For purposes of this invention, an effective amount of an adenoviral vector is an amount that is sufficient to palliate, ameliorate, stabilize, reverse, slow or delay the progression of the disease state.

“Expression” includes transcription and/or translation.

As used herein, the term “comprising” and its cognates are used in their inclusive sense; that is, equivalent to the term “including” and its corresponding cognates.

As used herein, “isolated” refers to a polypeptide or nucleic acid that is removed from at least one component with which it is naturally associated.

“A,” “an” and “the” include plural references unless the context clearly dictates otherwise.

Adenovirus Sequences

At least 47 serotypes of human adenoviruses have been described. Reviews of the most common serotypes associated with particular diseases have been published. See for example, Foy H. M. (1989) Adenoviruses In Evans AS (ed). Viral Infections of Humans. New York, Plenum Publishing, pp 77-89 and Rubin B. A. (1993) Clinical picture and epidemiology of adenovirus infections, Acta Microbiol. Hung 40:303-323. The capsid of a human adenovirus demonstrates icosahedral symmetry and contains 252 capsomers. The capsomers consist of 240 hexons and 12 pentons with a projecting fiber on each of the pentons. The pentons and hexons are each derived from different viral polypeptides. The fibers, which are responsible for type-specific antibodies, vary in length among human strains. The hexons are group specific complement-fixing antibodies, whereas the pentons are especially active in hemgglutination (Plotkin and Orenstein, Vaccines, 3rd edition, W. B. Saunders Company Philadelphia, pp609-623). The fiber region assumes a homotrimeric conformation which is necessary for association of the mature fiber protein with the penton base in the formation of the adenovirus capsid. Fiber associates with penton base by virtue of non-covalent interactions between the amino terminus of the fiber trimer and a conserved domain within the penton base. It has been shown that the globular carboxyterminal knob domain of the adenovirus fiber protein is the ligand for attachment to the adenovirus primary cellular receptor (Krasnykh et al. (1996) Journal of Virology, 70:6839.). The distal, C-terminal domain of the trimeric fiber molecule terminates in a knob which binds with high affinity to a specific primary receptor. After binding, Arg-Gly-Asp (RGD) motifs in the penton base interact with cellular integrins which function as secondary receptors. This interaction triggers cellular internalization whereby the virion resides within the endosome. The endosome membrane is lysed in a process mediated by the penton base, releasing the contents of the endosome to the cytoplasm. During these processes, the virion is gradually uncoated and the adenovirus DNA is transported into the nucleus (Shayakhmetov et al. (2000) Journal of Virology 74:2567-2583). Human adenoviruses Ad3, Ad4, Ad5, Ad9 and Ad35 are available from the American Tissue Culture Collection ATCC). The National Center for Biotechnology Information GenBank accession number for Ad5 is M73260/M29978; for Ad9×74659; and for Ad35, U10272. Chow et al. (1977, Cell 12:1-8) disclose human adenovirus 2 sequences; Davison et al. (1993, J Mole. Biol. 234:1308-1316) disclose the DNA sequence of human adenovirus type 40; Sprengel et al. (1994, J. Virol. 68:379-389) disclose the DNA sequence for human adenovirus type 12 DNA. As described in WO 02/096939, the human Ad5 pIX gene is present at the left end of the Ad5 adenoviral genome positioned between E1 B and E2 regions, e.g. from about nucleotides 3609 to about 4031. Human adenovirus capsid protein 1×sequences are disclosed in PCT publication WO 01/58940.

The bovine adenoviruses (BAV) comprise at least nine serotypes divided into two subgroups. These subgroups have been characterized based on enzyme-linked immunoassays (ELISA), serologic studies with immunofluorescence assays, virus-neutralization tests, immunoelectron microscopy, by their host specificity and clinical syndromes. Subgroup 1 viruses include BAV 1, 2, 3 and 9 and grow relatively well in established bovine cells compared to subgroup 2 which includes BAV 4, 5, 6, 7 and 8.

BAV3 was first isolated in 1965 and is the best characterized of the BAV genotypes, containing a genome of approximately 35 kb (Kurokawa et al (1978) J. Virol. 28:212-218). Reddy et al. (1999, Journal of Virology, 73: 9137) disclose a replication-defective BAV3 as an expression vector. BAV3, a representative of subgroup 1 of BAVs (Bartha (1969) Acta Vet. Acad. Sci. Hung. 19:319-321), is a common pathogen of cattle usually resulting in subclinical infection (Darbyshire et al. (1965). J. Comp. Pathol. 75:327-330), though occasionally associated with a more serious respiratory tract infection (Darbyshire et al., 1966 Res. Vet. Sci. 7:81-93; Mattson et al., 1988 J. Vet Res 49:67-69). Like other adenoviruses, BAV3 is a non-enveloped icosahedral particle of 75 nm in diameter (Niiyama et al. (1975) J. Virol. 16:621-633) containing a linear double-stranded DNA molecule. BAV3 can produce tumors when injected into hamsters (Darbyshire, 1966 Nature 211:102) and viral DNA can efficiently effect morphological transformation of mouse, hamster or rat cells in culture (Tsukamoto and Sugino, 1972 J. Virol. 9:465-473; Motoi et al., 1972 Gann 63:415-418). Cross hybridization was observed between BAV3 and human adenovirus type 2 (HAd2) (Hu et al., 1984 J. Virol. 49:604-608) in most regions of the genome including some regions near but not at the left end of the genome. Reddy et al. (1998, Journal of Virology, 72:1394) disclose nucleotide sequence, genome organization, and transcription map of BAV3. Reddy et al. (1998, Journal of Virology, supra) disclose nucleotide sequences for BAV3. In the polynucleotide sequence for BAV3, the penton regions starts at 12919 and ends at 14367; the hexon region starts at 17809 and ends at 20517; the fiber region starts at 27968 and ends at 30898. The knob region (or domain) of the fiber protein starts after the residues TLWT motif. A transcriptional map for BAV3 is disclosed in U.S. published patent application US 2002-0034519A1, specifically incorporated herein by reference. BAV3 capsid piX region is disclosed in Zheng et al. (1994, Virus Research 31:163-186). BAV-3 capsid proteins are encoded by the following nucleotide (nt) ranges based on GenBank accession number AF030154: pIX nt 3,200 to 3,577; IIIa nt 11,098 to 12,804; VI nt 16,871 to 17,662 and VIII nt 25803 to 26453. Additional bovine adenovirus capsid proteins are known in the art. Bovine adenovirus expression systems have been disclosed in U.S. Pat. No. 5,820,868, issued Oct. 31, 1998; 6,319,716, issued Nov. 20, 2001; and U.S. published patent application US 2002-0034519A1.

Nucleotide sequences have been determined for segments of the genome of various PAV serotypes. Sequences of the E3, pVIII and fiber genes of PAV-3 were determined by Reddy et al. (1995) Virus Res. 36:97-106. The E3, pVIII and fiber genes of PAV-1 and PAV-2 were sequenced by Reddy et al. (1996) Virus Res. 43:99-109, while the PAV-4 E3, pVIII and fiber gene sequences were determined by Kleiboeker (1994) Virus Res. 31:17-25. The PAV-4 fiber gene sequence was determined by Kleiboeker (1995) Virus Res. 39:299-309. Inverted terminal repeat (ITR) sequences for all five PAV serotypes (PAV-1 through PAV-5) were determined by Reddy et al. (1995) Virology 212:237-239. The PAV-3 penton sequence was determined by McCoy et al. (1996) Arch. Virol. 141:1367-1375. The nucleotide sequence of the E1 region of PAV-4 was determined by Kleiboeker (1995) Virus Res. 36:259-268. The sequence of the protease (23K) gene of PAV-3 was determined by McCoy et al. (1996) DNA Seq. 6:251-254. The sequence of the PAV-3 hexon gene (and the 14 N-terminal codons of the 23K protease gene) has been deposited in the GenBank database under accession No. U34592. The sequence of the PAV-3 100K gene has been deposited in the GenBank database under accession No. U82628. The sequence of the PAV-3 E4 region has been determined by Reddy et al. (1997) Virus Genes 15:87-90. Cross-neutralization studies have indicated the existence of at least five serotypes of PAV. See Derbyshire et al. (1975) J. Comp. Pathol. 85:437-443; and Hirahara et al. (1990) Jpn. J. Vet. Sci. 52:407-409. Previous studies of the PAV genome have included the determination of restriction maps for PAV Type 3 (PAV-3) and cloning of restriction fragments representing the complete genome of PAV-3. See Reddy et al. (1993) Intervirology 36:161-168. In addition, restriction maps for PAV-1 and PAV-2 have been determined. See Reddy et al. (1995b) Arch. Virol. 140:195-200. PCT publication WO 99/53047 published Oct. 21, 1999, and U.S. Pat. No. 6,492,343, provides a transcriptional map of PAV3, specifically incorporated herein by reference. Specifically, the transcriptional start site of the PAV3 pIX gene is located at polynucleotide 3377 (with the ATG at 3394) and the polyA is located at polynucleotide 4085, with respect to the PAV3 sequence disclosed in Reddy et al. (1998, Virology, 251:414-426).

Vrati et al. 1995 (Virology, 209:400-408) and Vrati et al.1996 (Virology, 220:186) disclose sequences for ovine adenovirus.

The present invention provides chimeric adenovirus capsid proteins, and vectors expressing them, in particular adenovirus vectors, wherein said chimeric adenovirus capsid protein comprises a part of or all of at least one adenovirus capsid protein and a binding partner of a cell-surface binding site present in a cell of gut associated lymphoid tissues (GALT) of a mammal, wherein the adenovirus capsid protein is located on the surface of the adenovirus capsid and the chimeric adenovirus capsid protein is capable of binding the cell present in gut associated lymphoid tissues (GALT). Vectors comprising such chimeric adenovirus capsid proteins, in particular adenovirus vectors, are particularly advantageous for the delivery of proteins or antigens to the GALT of mammals and as oral vaccines when compared to a comparable adenovirus vectors lacking a binding partner of a cell-surface binding site present in a cell of gut associated lymphoid tissues (GALT) of a mammal. Without being bound by theory, vectors, in particular adenovirus vectors, comprising chimeric adenovirus capsid proteins of the present invention that are capable of binding cells present in the GALT by virtue of the presence of a binding partner for a cell-surface specific site on a cell in GALT, are particularly advantageous for use as oral vaccines for ruminant mammals, such as cows and sheep, due to the reduction in digestive tract degradation of the vector as compared to a comparable vector lacking a binding partner. In other examples, a vector comprising a polynucleotide encoding a chimeric adenovirus capsid protein comprising a binding partner allows for purification of the vector, such as by adsorbing the vector to a substrate for the binding partner. Such methods are known in the art.

The present invention encompasses the use of any adenovirus capsid protein as long as the protein is located on the surface of the capsid or is capable of displaying a binding partner on the surface of the capsid. The present invention encompasses a chimeric adenovirus capsid protein comprising a part of an adenovirus capsid protein, as long as an adenovirus vector comprising nucleic acid encoding the chimeric adenovirus capsid protein is capable of forming an adenovirus capsid. Accordingly, the present invention provides an adenovirus capsid containing a chimeric capsid protein, and host cells and compositions comprising the adenovirus capsid. The present invention encompasses complexes comprising a chimeric adenovirus capsid protein bound to a cell present in GALT tissue. An adenovirus capsid can be further modified to express additional proteins or other entities, such as carbohydrates for example, that are capable of binding heterologous species cells and therefore allow for binding of an adenovirus encompassed within the present invention to a cell of a heterologous species. For example, an adenovirus based vaccine composition expressing an antigen of a human pathogen and expressing a bovine adenovirus capsid protein in fusion with a cell-surface binding site of a human cell in GALT can be further genetically engineered to express an entity that specifically binds human cells, that is a human cell-surface binding site. Such an adenovirus vector would provide the advantage of providing for targeted delivery of a human antigen to human cells present in GALT tissue by a bovine adenovirus, thereby reducing the potential for production of neutralizing antibodies seen with the use of human adenovirus. A vector comprising a chimeric adenovirus capsid protein can be further modified to express addition proteins, such as antigens of pathogens, in particular for vaccine purposes. In some examples, the chimeric adenovirus capsid protein and vectors expressing the protein are produced by recombinant DNA technology, by providing nucleic acid encoding the chimeric adenovirus capsid protein. A part of or all of nucleic acid encoding a chimeric adenovirus capsid protein can be synthesized by chemical means known to those of skill in the art. Such nucleic acid can be ligated in vitro to a vector. In other examples, a binding partner for a cell-surface binding site of a cell in GALT is covalently bound or conjugated or linked to an adenovirus capsid protein located on the surface of the capsid and in some examples, after formation of the adenovirus capsid. A binding partner can be directly bound, conjugated or linked to an adenovirus capsid protein or indirectly bound, conjugated or linked to the adenovirus capsid protein, such as by use of a spacer. An adenovirus capsid protein, such as a fiber protein or pIX protein, may be linked together with a binding partner by any of the conventional ways of cross-linking polypeptides, such as those generally described in O'Sullivan et al. (1979, Anal. Biochem. 100:100-108). For example, the binding partner maybe enriched with thiol groups and the molecule on the surface of the virus or virus-like particle, e.g. the pIX protein, may be reacted with a bifunctional agent capable of reacting with those thiol groups, for example with the N-hydroxysuccinimide ester of iodoacetic acid (NE11A) or N-succinimidyl (2-pyridyldithio)propionate (SPDP). Amide and thioether bonds, for example achieved with m-maleimidobenzoyl-N-hydroxysuccinimide ester, are generally more stable in vivo than disulphide bonds. Other chemical procedures may be useful in joining oligosaccharide and lipids to polypeptides. Covalent coupling between the binding partner and the capsid protein may also be performed using a polymer such as polyethylene glycol (PEG) or its derivatives (see for example WO99/40214; Bioconjugate Techniques, 1996, 606-618; ed G Hermanson; Academic Press and Frisch et al., 1996, Bioconjugate Chem. 7, 180-186). The binding partner and the capsid protein may also be non-covalently coupled, for example via electrostatic interactions or through the use of affinity components such as protein A, biotin/avidin, which are able to associate both partners. Immunological coupling can also be used in the context of the present invention, for example using antibodies to conjugate the binding partner to the capsid protein. For example, it is possible to use biotinylated antibodies directed to a capsid protein on the surface of the capsid and streptavidin-labelled antibodies directed against the binding partner according to the technique disclosed by Roux et al. (1989, Proc. Natl. Acad Sci USA 86, 9079). Bifunctional antibodies directed against each of the coupling partners are also suitable for this purpose. In some examples, a binding partner is conjugated to an adenovirus capsid after formation of the adenovirus capsid.

In some examples disclosed herein, a chimeric adenovirus capsid protein comprises the adenovirus capsid protein IX (pIX). The term adenovirus capsid “pIX” is well understood in the art and refers to a pIX protein encoded by an adenoviral genome which is known to be integrated into the capsid of virus or virus-like particles. An adenovirus capsid pIX can be isolated from an adenovirus genome, such as those described herein, by conventional recombinant methods and can be from any source. In examples illustrated herein, the adenovirus capsid pIX is obtainable from BAV3. Additional adenovirus capsid pIX can be identified based on nucleotide and amino acids disclosed herein and available in public databases. In other examples, the adenovirus capsid protein is adenovirus fiber protein. In some examples, the binding partner of a chimeric capsid protein is present within or at the N-terminus of the adenovirus capsid protein, present within or at the C-terminus of the adenovirus capsid protein, or present internal to the adenovirus capsid protein (as long as the chimeric capsid protein is capable of displaying the binding partner on the surface of the capsid). For capsid proteins embedded in the virion capsid, such as the N-terminal domain, the use of linkers can be used to facilitate display of the binding partner on the surface of the capsid. The present invention encompasses a chimeric adenovirus capsid protein comprising part of an adenovirus capsid protein, as long as an adenovirus vector comprising nucleic acid encoding the chimeric adenovirus capsid protein is capable of forming an adenovirus capsid. WO 02/096939 discloses that human adenovirus capsid pIX is highly conserved at the N-terminus and, without being bound by theory, may be essential for the capsidic structural properties of the capsid. WO 02/096939 discloses that the human adenovirus capsid pIX C-terminus comprises leucine-repeats, and without being bound by theory, appears critical for transactivating function and can be modified without altering the structural function of pIX. In some examples, an adenovirus capsid pIX protein is modified such that the binding partner is located within or at the C-terminus and in some examples, precedes the C-terminus by about 40 amino acids, about 30 amino acids, about 20 amino acids, about 10 amino acids or about 5 amino acids. The insertion can be between residues or can replace residues of pIX. In other examples, an adenovirus capsid pIX protein is modified such that the binding partner is located within or at the N-terminus and in some examples, precedes the N-terminus by about 40 amino acids, about 30 amino acids, about 20 amino acids, about 10 amino acids or about 5 amino acids. The insertion can be between residues or can replace residues of pIX. As disclosed in WO 01/58940, specifically incorporated herein by reference, additional modifications can be made to an adenovirus vector comprising a chimeric adenovirus capsid protein in order to reduce the ability of the mammalian host to develop neutralizing antibodies. See Zakhartchouk et al., 2004, Virology, vol. 320:291-300, specifically incorporated herein by reference, which discloses BAV-3 containing heterologous protein in the C-terminus of pIX.

The present invention encompasses the use of encapsidation systems which comprise a vector and optionally a helper cell that comprise adenovirus sequences necessary to form an adenovirus capsid and may or may not comprise viral proteins. For example, viral proteins essential for adenovirus replication, or adenovirus encapsidation for example, may be provided by a helper cell or may be provided on a vector. The vector may be a replication-competent adenovirus vector that comprises nucleic acid sequences necessary for viral replication and may comprise nucleic acid sequences necessary for encapsidation; a replication-deficient adenovirus vector lacking sequences necessary for replication, such as for example, nucleic acid encoding E1 function (that is, E1 functional protein), that requires a helper cell that expresses the E1 function for replication; or a vector that comprises adenovirus sequences necessary for encapsidation wherein adenoviral proteins are provided by a helper cell or a vector that comprises adenoviral proteins wherein adenovirus sequences necessary for encapsidation are provided by a helper cell. In all examples, the vector may further comprise heterolgous nucleic acid encoding a protein, such as an antigen of a pathogen.

Binding Partners

Binding partners of cells present in the GALT are molecules or entities that are capable of binding a cell-surface binding site of a cell present in GALT tissue and include, but are not limited to cell-binding proteins, including lectins, or peptides (including modified proteins and peptides, such as for example, glycoproteins and mucoproteins); amino acids motifs, or short stretches of amino acids, such as those constituting a peptide hormone; domains of polypeptides that can fold independently into a structure that can bind a target cell; carbohydrates, including oligosaccharides; lipids; mucin molecules; antibodies, including but not limited to monoclonal antibody, or a fragment thereof capable of binding to a cell-surface binding site of a cell, a single chain of an antibody, a single domain antibody or a minimal recognition unit of an antibody. In some examples, a chimeric adenovirus capsid protein comprises a binding partner present on the surface of the adenovirus capsid, thereby allowing for binding of the chimeric capsid protein to the cell-surface binding site of the target cell. As used herein, a “target cell” is a cell wherein introduction and/or infection of a vector or virus is desired. As used herein, “target cells of GALT tissues” include, but are not limited to, cells of Peyer's patches, epithelium cells of any tissue of GALT, including epithelium cells of Peyer's patches (PP), and M cells of Peyer's patches. In some examples, the target cell is a mammalian jejunal PP cell.

Peyer's patches comprise transmucosal clusters of lymphoid follicles overlaid with a specialized lympho-epithelium and play a central role in the induction of mucosal immune responses in the gut. (Makala et al. 2002, Pathobiology, 70:55-68) This epithelium plays an important role in immune protection by delivering small samples of luminal material to organized mucosal lymphoid tissues that function as sites for initiation of mucosal immune responses. Lymphoid follicles at these sites are covered by follicle-associated epithelium containing microfold (M) cells, a unique cell type specialized for transepithelial transport of particles and macromolecules. The M cells serve as a portal of entry for antigens and pathogens. (Giannasca et al., 1994, Am. J. Physiol. 267 (Gastrointest. Liver Physiol. 30): G1108-G1121). The present invention encompasses chimeric adenovirus capsid proteins (and vectors capable of expressing such chimeric adenovirus capsid proteins, in particular viral vectors, such as adenovirus vectors) that comprise binding partners of proteins or other structures, such as for example carbohydrates, on the cells of the Peyer's patches of GALT, in particular epithelial cells and M cells. Adenovirus vectors expressing the chimeric adenovirus capsid proteins and expressing heterologous proteins, such as antigens of pathogens, can be replication-competent or replication-defective. In some examples, where a binding partner binds Peyer's patches M cells, which serve as portals of entry of antigens to the immune system, a replication-deficient or replication-competent adenovirus is used. In other examples where a binding partner binds other tissues of the GALT or Peyer's patches, such as epithelial cells, a replication-competent adenovirus is used to produce more viral particles in the vicinity of the M cells. Such adenovirus vectors and viral particles comprising such vectors are used for the delivery of the vectors, and the antigens they express, to mammals, in particular mammalian ruminants, in particular for the delivery of oral vaccines.

Giannasca et al. (1994, Am. J. Physiol. 267 (Gastrointest. Liver Physiol. 30): G1108-G1121) specifically incorporated herein by reference, describe glycoconjugates of intestinal M cells in mice. Briefly, lectins and antibody probes (shown in Table 1) were used in immunohistochemical analysis of sections of mouse intestine. Table 2 shows the lectin/antibody binding patterns in mouse Peyer's patch epithelium, including M cells. As demonstrated in Giannasca et al., α(1-2)-fucose-specific lectins Ulex europaeus type I (UEA) (Pereira, 1978, Arch. Biochem. Biophys. 185:108-115); Anguilla anguilla (AAA) (Kelly, 1984, Biochem. J. 220: 221-226); and Lotus tetragonolobus (LTA) (Pereira, 1974, Biochemistry 13:3184-3192) bound selectively to M cells in the follicle associated epithelium (FAE). The identification of these molecules unique to M cells are used to develop binding partners, such as antibodies that target M cells of mammalian Peyer's patches, by conventional means.

Gebert et al. (1994, Cell Tissue Res. 276: 213-221) specifically incorporated herein by reference, describe Cytokeratin 18 as an M-cell marker in porcine Peyer's patches. Gebert et al. describe cytokeratin 18 antibodies, CK5, CY90 amd KS-B17.2 (all available from Sigma). The identification of this molecule that binds porcine M cells is used to develop binding partners, such as antibodies that target M cells of mammalian Peyer's patches, by conventional means.

Pappo et al. (1989, Cellular Immunology, 120:31-41) specifically incorporated herein by reference, disclose the generation and characterization of monoclonal antibodies recognizing follicle epithelial M cells in Rabbit GALT. Pappo et al. 1989, supra, disclose that monoclonal antibodies 5D9 and 5B11 recognize phagocytic M cells from FAE of rabbits.

As described herein in the examples, a monoclonal antibody against epithelial cells of the small intestine of sheep was generated that cross-reacted with bovine and porcine jejunum Peyer's patches (PP), ileal PP and jejunum tissues. The present invention encompasses chimeric adenovirus capsid proteins, encapsidation systems capable of expressing a chimeric adenovirus capsid and vectors, such as adenovirus vectors, expressing the chimeric adenovirus capsid proteins, that comprise a part of or all of at least one adenovirus capsid protein and a monoclonal antibody against sheep intestinal epithelial cells, or a binding fragment thereof. In some examples, the monoclonal antibody is cross reactive with a cell-surface binding site in GALT tissue of bovine, ovine and porcine mammals. Such an antibody provides the advantage of having one binding partner that cross reacts with a cell in GALT of several mammalian species. In some examples, an adenovirus vector expresses a chimeric adenovirus capsid protein that comprises a part of or all of adenovirus capsid protein 1× and a fragment of a monoclonal antibody against epithelial cells of the small intestine of sheep. In some examples, the binding partner is a single chain antibody, and in some examples, a single chain antibody based on a monoclonal antibody against cells present in GALT of a mammal that that cross-reacts with additional mammalian species GALT. Single chain antibodies are produced based on methods known in the art including, for example, Euggan et al. (2001, Virol. Immunology, 14:263), specifically incorporated herein by reference.

Binding of a particular chimeric adenovirus capsid protein to GALT tissues of a mammal is assayed by any means known in the art including histochemical staining, such as immunohistochemical staining. For example, cross sections of mammalian GALT tissue identified to contain a cell of interest, such as an epithelial cell of GALT or an M cell, is exposed to an appropriately labeled chimeric adenovirus capsid protein under suitable conditions. Binding of the chimeric adenovirus capsid protein to a particular cell is detected. A cross section of mammalian GALT is provided in Gebert et al., International Review of Cytology, vol. 167, supra. Additionally, antibodies to cell markers or proteins found on cells present in the GALT, including monoclonal antibodies or binding fragments thereof, are made by means well known to those of skill in the art and disclosed herein.

Production of Chimeric Adenovirus Capsid Proteins

In some examples disclosed herein, a chimeric adenovirus capsid protein is produced by recombinant DNA techniques. The present invention provides vectors such as adenovirus vectors comprising polynucleotides encoding chimeric adenovirus capsid proteins. Molecular cloning and viral construction are generally known in the art. In some examples, an adenovirus vector comprising nucleic acid encoding an adenovirus capsid protein is ligated in vitro to nucleic acid encoding a binding partner and subsequently introduced into a host cell by means known in the art. In some examples, a recombinant adenovirus vector comprising a polynucleotide encoding a chimeric adenovirus capsid protein and/or a transgene is constructed by in vivo recombination between a plasmid and an adenoviral genome. Generally, transgenes are inserted into a plasmid vector containing a portion of the desired adenovirus genome, and in some examples, the adenovirus genome may possess a mutation of, for example, a deletion of one or more adenoviral sequences encoding viral proteins. In some examples, adenovirus sequences encoding protein function essential for viral replication, such as the E1 region, are mutated, such as for example, deleted in part or all of the E1 sequence. In other examples, the adenovirus is replication-competent. The transgene is inserted into the adenovirus insert portion of the plasmid vector, such that the transgene is flanked by adenovirus sequences that are adjacent on the adenovirus genome. The adenovirus sequences serve as “guide sequences,” to direct insertion of the transgene to a particular site in the adenovirus genome; the insertion site being defined by the genomic location of the guide sequences. Mammalian adenovirus packaging sequences can be added into an adenovirus vector by means known to those of skill in the art.

The plasmid vector is generally a bacterial plasmid, allowing multiple copies of the cloned sequence to be produced. In one embodiment, the plasmid is co-transfected, into an appropriate host cell, with an adenovirus genome, or portion thereof. The adenovirus genome can be isolated from virions, or can comprise a genome that has been inserted into a plasmid, using standard techniques of molecular biology and biotechnology. In some examples, adenovirus vector sequences can be deleted in regions such as, for example, E1, E3, E4 and/or the region between E4 and the right end of the genome and/or late regions such as L1-L5. Adenovirus genomes can be deleted in essential regions, such as E1, if the essential function are supplied by a helper cell line. Porcine E1A, E1B^{large} and E4 ORF3 have been determined to be essential for viral replication of PAV3. For PAV3, the E1A region is from nucleotide 533 to nucleotide 1222, with respect to the PAV3 sequence disclosed in Reddy et al. 1998, Virology 251:414-426, the E1B^{small} region is from nucleotide 1461 to nucleotide 2069 with respect to Reddy et al. supra, and the E1B^{large} region is from nucleotide 1829 to nucleotide 3253 of Reddy et al. supra. E1B^{small} and E1B^{large} nucleotide regions are overlapping and are differentially transcribed. Depending upon the intended use of a PAV vector, PAV constructs can be made comprising a deletion of a part of or all of the E1B^{small} region. For example, if the entire E1B function is intended to be deleted, the entire E1B nucleotide region from nucleotides 1461 to 3253 can be deleted; or the region from nucleotides 1461 to 2069 can be deleted (which disrupts both E1B^{small} and E1B^{large} function); or the region from 1461 to 2069 and additionally, any portion of nucleotides 2069 through 3253 can be deleted. If it is intended to delete E1B^{small} nucleotides while retaining E large function, nucleotides 1461 to 1829 are deleted, leaving the nucleotide region for E large intact. It has been determined that PAV E4 ORF3 is essential for replication. PAV E4 ORF3 is from between about nt 32656 to nt 33033 of the PAV sequence shown in Reddy et al. supra. In some examples, the adenovirus vector is deleted in multiple nucleic acid sequences encoding viral proteins as long as any sequences essential for replication are provided by a helper virus.

Insertion of a cloned transgene into a viral genome can occur by in vivo recombination between a plasmid vector (containing transgene sequences flanked by adenovirus guide sequences) and an adenovirus genome following co-transfection into a suitable host cell. The adenovirus genome contains inverted terminal repeat (ITR) sequences required for initiation of viral DNA replication (Reddy et al. (1995), Virology 212:237-239). Incorporation of the cloned transgene into the adenovirus genome thus places the transgene sequences into a DNA molecule containing adenoviral sequences.

Incorporation of the cloned transgene into an adenovirus genome places these sequences into a DNA molecule that can be replicated and packaged in an appropriate helper cell line, such as a helper cell line that expresses adenovirus functions essential for replication if the adenovirus is replication-deficient. Multiple copies of a single transgene sequence can be inserted to improve yield of the gene product, or multiple transgene sequences can be inserted so that the recombinant virus is capable of expressing more than one heterologous gene product. The transgene sequences can contain additions, deletions and/or substitutions to enhance the expression and/or immunological effect of the expressed gene product(s).

Attachment of guide sequences to a heterologous sequence can also be accomplished by ligation in vitro. In this case, a nucleic acid comprising a transgene sequence flanked by an adenovirus guide sequences can be co-introduced into a host cell along with the adenovirus genome, and recombination can occur to generate a recombinant adenovirus vector. Introduction of nucleic acids into cells can be achieved by any method known in the art, including, but not limited to, microinjection, transfection, electroporation, CaPO₄ precipitation, DEAE-dextran, liposomes, particle bombardment, etc.

In one embodiment of the invention, a recombinant adenovirus expression cassette can be obtained by cleaving a wild-type adenovirus genome with an appropriate restriction enzyme to produce an adenovirus restriction fragment representing a portion of the genome. The restriction fragment can be inserted into a cloning vehicle, such as a plasmid, and thereafter at least one transgene sequence (which may or may not encode a foreign protein) can be inserted into the adenovirus region with or without an operatively-linked eukaryotic transcriptional regulatory sequence. The recombinant expression cassette is contacted with the adenovirus genome and, through homologous recombination or other conventional genetic engineering methods, the desired recombinant is obtained. These DNA constructs can then undergo recombination in vitro or in vivo, with an adenovirus genome either before or after transformation or transfection of an appropriate host cell.

Deletion of adenovirus sequences, to provide a site for insertion of heterologous sequences or to provide additional capacity for insertion at a different site, or addition of sequences, such as an adenovirus E3 gene region, can be accomplished by methods well-known to those of skill in the art. For example, for adenovirus sequences cloned in a plasmid, digestion with one or more restriction enzymes (with at least one recognition sequence in the adenovirus insert) followed by ligation will, in some cases, result in deletion of sequences between the restriction enzyme recognition sites. Alternatively, digestion at a single restriction enzyme recognition site within the adenovirus insert, followed by exonuclease treatment, followed by ligation will result in deletion of adenovirus sequences adjacent to the restriction site. A plasmid containing one or more portions of the adenovirus genome with one or more deletions, constructed as described above, can be co-transfected into a bacterial cell along with a plasmid containing a full-length adenovirus genome to generate, by homologous recombination, a plasmid containing a adenovirus genome with a deletion at a specific site. Adenovirus virions containing the deletion (or addition) can then be obtained by transfection of appropriate mammalian cells, such as for example, mammalian cells comprising complementing adenovirus nucleotide sequences deleted from the adenovirus vector, with the plasmid containing an adenovirus genome with a deletion at a specific site.

Expression of an inserted sequence in a recombinant adenovirus vector will depend on the insertion site. Accordingly, insertion sites may be adjacent to and downstream (in the transcriptional sense) of adenovirus promoters. Locations of restriction enzyme recognition sequences downstream of adenovirus promoters, for use as insertion sites, can be easily determined by one of skill in the art from the adenovirus nucleotide sequences known in the art Alternatively, various in vitro techniques can be used for insertion of a restriction enzyme recognition sequence at a particular site, or for insertion of heterologous sequences at a site that does not contain a restriction enzyme recognition sequence. Such methods include, but are not limited to, oligonucleotide-mediated heteroduplex formation for insertion of one or more restriction enzyme recognition sequences (see, for example, Zoller et al. (1982) Nucleic Acids Res. 10:6487-6500; Brennan et al. (1990) Roux 's Arch. Dev. Biol. 199:89-96; and Kunkel et al. (1987) Meth. Enzymology 154:367-382) and PCR-mediated methods for insertion of longer sequences. See, for example, Zheng et al. (1994) Virus Research 31:163-186.

In additional examples, an adenovirus vector may further comprise an intron 5′ to a heterologous transgene, wherein said vector is capable of expressing greater levels of the heterologous transgene than a comparable adenovirus vector comprising a heterologous transgene and lacking an intron 5′ to said heterologous transgene. The use of introns in adenovirus systems is disclosed in US patent application publication 2002-0064859, hereby incorporated by reference in its entirety.

It is also possible to obtain expression of a transgene or heterologous nucleic acid sequence inserted at a site that is not downstream from an adenovirus promoter, if the heterologous sequence additionally comprises transcriptional regulatory sequences that are active in eukaryotic cells. Such transcriptional regulatory sequences can include cellular promoters such as, for example, the bovine hsp70 promoter and viral promoters such as, for example, herpesvirus, adenovirus and papovavirus promoters and DNA copies of retroviral long terminal repeat (LTR) sequences.

In another example, homologous recombination in a procaryotic cell can be used to generate a cloned adenovirus genome; and the cloned adenovirus genome can be propagated as a plasmid. Infectious virus can be obtained by transfection of mammalian cells with the cloned adenovirus genome rescued from plasmid-containing cells. Mammalian cells can also be transfected with adenovirus vectors.

Suitable host cells include any cell that will support recombination between an adenovirus genome and a plasmid containing adenovirus sequences, or between two or more plasmids, each containing adenovirus sequences. Recombination is generally performed in procaryotic cells, such as E. coli, while transfection of a plasmid containing a viral genome, to generate virus particles, is conducted in eukaryotic cells, such as mammalian cells, including bovine cell cultures, human cell cultures and porcine cell cultures. The growth of bacterial cell cultures, as well as culture and maintenance of eukaryotic cells and mammalian cell lines are procedures which are well-known to those of skill in the art. Accordingly, the present invention provides host cells comprising adenovirus vectors of the present invention. The present invention also provides viral particles comprising viral vectors.

In one example of the invention, replication-defective recombinant adenovirus vectors capable of expressing a chimeric adenovirus capsid protein are used for expression of a transgene, such as for example, an antigen of a pathogen. In some examples, the replication-defective adenovirus vector lacks E1 region function (that is, that lacks E1 functional protein). In other examples, the adenovirus vector lacks nucleic acid encoding multiple adenoviral genes. Transgene sequences can be inserted so as to replace deleted adenovirus region(s), and/or can be inserted at other sites in the genome. Replication-defective vectors with deletions in essential regions are grown in helper cell lines, which provide the deleted function.

Accordingly, the present invention provides recombinant helper cell lines, produced according to the present invention by constructing an expression cassette comprising an adenoviral region(s) necessary for complementation of adenovirus regions deleted in the adenovirus vector and transforming host cells therewith to provide complementing cell lines or cultures providing deleted functions. In some examples, the adenovirus vector lacks E1 regions essential for replication and the host cell is transformed with the adenovirus E1 region. The terms “complementing cell,” “complementing cell line,” “helper cell” and “helper cell line” are used interchangeably herein to denote a cell line that provides a viral function that is deficient in a deleted adenovirus vector. These recombinant complementing cell lines are capable of allowing a defective recombinant adenovirus to replicate and express one or more transgenes or fragments thereof.

More generally, replication-defective recombinant adenovirus vectors, lacking one or more essential functions encoded by the adenovirus genome, can be propagated in appropriate complementing cell lines, wherein a particular complementing cell line provides a function or functions that is (are) lacking in a particular defective recombinant adenovirus vector. Complementing cell lines can provide viral functions through, for example, co-infection with a helper virus, or by integrating or otherwise maintaining in stable form a fragment of a viral genome encoding a particular viral function. In another embodiment of the invention, adenovirus function can be supplied (to provide a complementing cell line) by co-infection of cells with a virus which expresses the function that the vector lacks. In another example, the present invention provides replication-competent adenovirus vectors capable of expressing a chimeric adenovirus capsid protein are used for expressing a transgene, such as a pathogen of an antigen.

The present invention encompasses vectors, such as adenovirus vectors that comprise one or more mammalian packaging domains. The main cis-acting packaging domains of BAV-3 have been identified localized between nucleotide position (nt) about 224 and about 540 relative to the left end of viral genome. Accordingly, the present invention encompasses vectors comprising bovine adenovirus encapsidation sequence. The present invention encompasses adenovirus vectors that comprise modifications in E 1 transcriptional control regions. BAV E1 transcriptional control regions including nucleotides from about 224 to about 382 relative to the left terminus of BAV-3 genome, which overlap the cis-acting packaging domains and nucleotides from about 537 to about 560 relative to the left terminus of BAV-3 genome. All BAV-3 nucleotides are with respect to the reference sequence GenBank accession number AF030154. The present invention encompasses BAV and BAV vectors comprising a modification of one or more E1 transcriptional control regions, wherein the modification can be a deletion and/or addition of part or all of one or more E1 transcriptional control regions. The present invention encompasses BAV and BAV vectors comprising part or all of one or more additional isolated bovine adenovirus E1 transcriptional control regions wherein the added sequence can be the same E1 transcriptional control region or a different E1 transcriptional control regions. The present invention encompasses BAV and BAV vectors comprising a deletion of part or all of an isolated bovine adenovirus E1 transcriptional control region.

It is predicted that left ITR of BAdV-3 contains core elements of the E1A promoter. DNA sequence analysis of the left ITR of BAV-3 showed the presence of a CCAAT box (nt 45-49), TATA-like box (nt 68-72), and most of GC boxes (nt 108-209). All BAV-3 nucleotides are with respect to the reference sequence GenBank accession number AF030154. The present invention encompasses the use of replication-competent bovine adenovirus. The present invention encompasses replication competent bovine adenovirus comprising the E1A promoter. In some examples, the replication-competent adenovirus comprises a deletion of non-essential gene regions (that is, regions of the genome that are non-essential for replication, such as part or all of E3 region) and comprises (retains) the essential E1 gene region along with the E1A promoter as described herein.

Six AT-rich motifs of PAV3 have been characterized which can provide the packaging ability to PAV3. The present invention provide recombinant vectors comprising PAV adenovirus sequences essential for encapsidation. PAV3 encapsidation sequences are shown in the tables below.

Table I provides a listing of the regions.

TABLE I
Alignment of Packaging sequences of PAV3

| | | | | |
|---|---|---|---|---|
| 233-237 | | CGG | AAATT | CCCGCACA |
| 264-268 | | GGG | ATTTT | GTGCCCTCT |
| 334-337 | | CGG | TATT | CCCCACCTG |
| 431-438 | | GTG | TATTTTTT | CCCCTCA |
| 449-454 | | GTG | TATATA | GTCCGCGC |
| 505-508 | | GAG | TTTT | CTCTCAGCG |
| 231-237 | GG | CGG | AAATT | CCCGCACA |
| 262-268 | GC | GGG | ATTTT | GTGCCCTCT |
| 332-337 | CC | CGG | TATT | CCCCACCTG |
| 429-438 | GG | GTG | TATTTTTT | CCCCTCA |
| 447-454 | CA | GTG | TATATA | GTCCGCGC |
| 503-508 | TA | GAG | TTTT | CTCTCAGCG |

PAV5 packaging domains are shown in Table II. PAV5 has six AT rich regions located between the left ITR (nt 1-154) and ATG (nt 418) of the E1A gene.

TABLE II
Alignment of expected packaging sequences of PAV5

| | | | |
|---|---|---|---|
| 187-192 | CTGG | TATTTT | CCAC |
| 207-211 | GTG | ATATT | GG |
| 217-220 | CC | TTTA | CCTGGG |
| 272-277 | CTC | AATTTTA | CCAC |
| 321-326 | GGTCG | ATTTTT | CCAC |
| 349-356 | CCC | TATTTATT | CTGCGCG |

The present invention encompasses adenovirus vectors comprising one or more PAV E1 transcriptional control regions. PAV E1 transcriptional control regions include nucleotides from about 252 to about 313; nucleotides from about 382 to about 433; nucleotides from about 432 to about 449; nucleotides from about 312 to about 382; nucleotides from about 312 to about 449; nucleotides from about 252 to about 449; and nucleotides from about 371 to about 432, all with respect to the PAV3 sequence disclosed in Reddy et al. 1998, Virology 251:414-426. The present invention encompasses PAV and PAV vectors comprising a modification of one or more E1 transcriptional control regions, wherein the modification can be a deletion or addition of part or all of one or more E1 transcriptional control regions. The present invention encompasses PAV and PAV vectors comprising part or all of one or more additional E1 transcriptional control regions wherein the added sequence can be the same E1 transcriptional control region or a different E1 transcriptional control regions.

Transgenes of Interest

The present invention encompasses adenoviral vectors comprising transgenes. The present invention encompasses vectors comprising heterologous nucleic acid sequences encoding protective determinants of various pathogens of mammals, including for example humans, cows, swine, sheep, or other mammals, for use in subunit vaccines and nucleic acid immunization. Representative human pathogen antigens include but are not limited to HIV virus antigens and hepatitis virus antigens. Representative swine pathogen antigens include, but are not limited to, pseudorabies virus (PRV) gp50; transmissible gastroenteritis virus (TGEV) S gene; genes of porcine respiratory and reproductive syndrome virus (PRRS), in particular ORFs 3, 4 and 5; genes of porcine epidemic diarrhea virus; genes of hog cholera virus; genes of porcine parvovirus; and genes of porcine influenza virus. Representative bovine pathogen antigens include bovine herpes virus type 1; bovine diarrhea virus; and bovine coronavirus. This list is not restrictive, and any other transgene of interest can be used in the context of the present invention. In some cases the gene for a particular antigen can contain a large number of introns or can be from an RNA virus, in these cases a complementary DNA copy (cDNA) can be used. It is also possible that only fragments of nucleotide sequences encoding proteins can be used (where these are sufficient to generate a protective immune response or a specific biological effect) rather than the complete sequence as found in the wild-type organism. Where available, synthetic genes or fragments thereof can also be used. However, the present invention can be used with a wide variety of genes, fragments and the like, and is not limited to those set out above. Adenovirus vectors can be used to express antigens for provision of, for example, subunit vaccines. Antigens used in the present invention can be either native or recombinant antigenic polypeptides or fragments. They can be partial sequences, full-length sequences, or even fusions (e.g., having appropriate leader sequences for the recombinant host, or with an additional antigen sequence for another pathogen). Antigenic polypeptide to be expressed by the virus systems of the present invention may contain full-length (or near full-length) sequences encoding antigens or, shorter sequences that are antigenic (i.e., encode one or more epitopes) can be used. The shorter sequence can encode a “neutralizing epitope,” which is defined as an epitope capable of eliciting antibodies that neutralize virus infectivity in an in vitro assay. The peptide can encode a “protective epitope” that is capable of raising in the host a “protective immune response;” i.e., a humoral (i.e. antibody-mediated), cell-mediated, and/or mucosal immune response that protects an immunized host from infection.

A gene of interest can be placed under the control of regulatory sequences suitable for its expression in a host cell. Suitable regulatory sequences are understood to mean the set of elements needed for transcription of a gene into RNA (ribozyme, antisense RNA or mRNA), for processing of RNA, and for the translation of an mRNA into protein. Among the elements needed for transcription, the promoter assumes special importance. It can be a constitutive promoter or a regulatable promoter, and can be isolated from any gene of eukaryotic, prokaryotic or viral origin, and even adenoviral origin. Alternatively, it can be the natural promoter of the gene of interest. Generally speaking, a promoter used in the present invention can be chosen to contain cell-specific regulatory sequences, or modified to contain such sequences. Promoters include, but are not limited to HSV-1 TK (herpesvirus type 1 thymidine kinase) gene promoter, the adenoviral MLP (major late promoter), in particular of human adenovirus type 2, the RSV (Rous Sarcoma Virus) LTR (long terminal repeat), the CMV (Cytomegalovirus) early promoter, and the PGK (phosphoglycerate kinase) gene promoter, for example, permitting expression in a large number of cell types.

Models of Mucosal Immune Response

The present invention encompasses chimeric adenovirus capsid proteins comprising a part of or all of at least one adenovirus capsid protein and a binding partner for a cell present in GALT of a mammal. Vectors comprising such chimeric adenovirus capsid proteins are used in vaccine compositions and methods, and in some examples, are used in oral vaccine compositions and methods for oral vaccine delivery to mammals, in particular ruminant mammals, such as cattle and sheep.

Gerdts et al. (2001, J. of Immunological Methods, 256: 19-33), specifically incorporated herein by reference, describe an in vivo intestinal loop model to analyze mucosal immune response. Briefly, a sterile intestinal segment is prepared in the jejunum of 4-6 month old lambs. This intestinal segment is subdivided into consecutive segments or “loops” that include a Peyer's patch (PP) or interspaces that lack visible PP. The functional integrity of M cell antigen uptake in the intestinal loops is evaluated by comparing the immune response induced by varying doses of antigens. Van der Lubben et al. (Journal of Drug Targeting, 10:449-456), specifically incorporated herein by reference, described a human intestinal M-cell model. Briefly, Caco-2 cells are co-cultured with human B-lymphocytes (Raji-cells) and cells which are morphologically and functionally similar to M-cells can be induced. Vectors, such as adenovirus vectors, comprising chimeric adenovirus capsid proteins, can be assayed in the Gerdts et al. supra, model or Van der Lubben et al. supra, model.

Uses of Adenovirus Vectors of the Present Invention

The use of viral vectors in therapeutic and prophylactic methods is well documented. The use of adenovirus vectors in oral vaccines for farm animals, especially ruminant mammals such as cows and sheep, has been problematic due to the presence of chambered stomachs and the degradation of the vector in the digestive tract. The present invention provides encapsidation systems, vectors, such as adenovirus vectors, and viral particles that express chimeric adenovirus capsid proteins that comprise a binding partner to cell-surface binding sites in cells present in GALT of a mammal. In some examples, vectors and adenovirus capsids encompassed within the present invention are degraded to a lesser extent in animal models, such as the Gerdts et al. supra, model disclosed herein, than comparable vectors lacking the binding partner. Accordingly, the present invention provides chimeric adenovirus capsid proteins, adenovirus vectors comprising nucleic acid encoding chimeric adenovirus capsid proteins, and encapsidation systems expressing adenovirus capsids that comprise a binding partner for a cell-surface binding site on a cell present in gut associated lymphoid tissues (GALT), in particular for targeted delivery of a protein to the GALT of a mammal. Such adenovirus vectors, encapsidation systems and adenovirus capsids are used to target delivery of an antigen, such as an antigen of a mammalian pathogen, to the GALT for the purpose of inducing a mucosal immune response to the antigen. In an illustrative example, the binding partner for a cell-surface binding site on a cell present in GALT is a monoclonal antibody that is cross reactive with (that is, that specifically binds) bovine, porcine and ovine jejunum Peyer's patches (PP). Such an antibody provides the advantage of having one binding partner that cross reacts with a cell in GALT of several mammalian species. In other examples, the binding partner specifically binds a cell-surface binding site on an GALT microfold (M) cell. The present invention provides viral vectors, such as adenovirus vectors, that express heterologous proteins, such as antigens of pathogens, that are targeted to cells in the GALT by virtue of the presence of a binding partner of a cell-surface binding site in cells present in GALT.

In examples wherein the binding partner binds an M cell present in GALT, which are specialized to deliver antigens to the immune system, a replication-defective or replication-competent adenovirus may be used. In examples wherein the binding partner binds an epithelial cell or a cell other than an M cell present in the GALT, a replication-competent adenovirus may be used, such that more viral particles are produced in the vicinity of the M cells. Any particular vaccination protocol may be designed to use a replication-deficient and/or replication-competent adenovirus irrespective of the target cell binding partner and includes for example, an initial vaccination with a replication-deficient adenovirus encompassed within the present invention with a boost vaccination with a replication-competent adenovirus encompassed within the present invention or the reverse.

An adenovirus vector of the present invention can be engineered to exhibit modified cell or tissue and species specificity. An adenovirus can be produced that expresses a capsid protein, such as for example, capsid protein IX (pIX) or a capsid fiber protein; a binding partner for a cell-surface binding site present on a cell in GALT tissue; and a molecule, protein, peptide or other entity that allows binding and/or entry of the adenovirus in a heterologous species cell. In an illustrative embodiment disclosed herein, BAV3 comprising capsid protein pIX modified to express an RGD motif is capable of transducing certain human cells.

The presence of a binding partner in an adenovirus vector comprising a chimeric adenovirus capsid protein can facilitate adenovirus purification methods, such as by affinity methods known in the art.

Also, the adenovirus vectors of the invention can be used for regulated expression of heterologous polypeptides encoded by transgenes. Standard conditions of cell culture, such as are known by those of skill in the art, will allow for expression of recombinant polypeptides. They can be used, in addition, for regulated expression of RNAs encoded by heterologous nucleotide sequences, as in for example, antisense applications and expression of ribozymes. The adenovirus vectors of the present invention capable of expressing a chimeric adenovirus capsid protein can be used for the expression of polypeptides in applications such as in vitro polypeptide production, vaccine production, nucleic acid immunization and gene delivery, for example such as antigens of pathogens. Polypeptides of therapeutic and/or diagnostic value include, but are not limited to, coagulation factors, growth hormones, cytokines, lymphokines, tumor-suppressing polypeptides, cell receptors, ligands for cell receptors, protease inhibitors, antibodies, toxins, immunotoxins, dystrophins, cystic fibrosis transmembrane conductance regulator (CFTR) and immunogenic polypeptides.

In some examples of the present invention adenovirus vectors will comprise heterologous sequences encoding protective determinants of various pathogens of mammals, including for example humans, cows, swine, sheep, or other mammals, for use in subunit vaccines and nucleic acid immunization. Representative human pathogen antigens include but are not limited to HIV virus antigens and hepatitis virus antigens. Representative swine pathogen antigens include, but are not limited to, pseudorabies virus (PRV) gp50; transmissible gastroenteritis virus (TGEV) S gene; genes of porcine respiratory and reproductive syndrome virus (PRRS), in particular ORFs 3, 4 and 5; genes of porcine epidemic diarrhea virus; genes of hog cholera virus; genes of porcine parvovirus; and genes of porcine influenza virus. Representative bovine pathogen antigens include bovine herpes virus type 1; bovine diarrhea virus; and bovine coronavirus.

Various foreign genes or nucleotide sequences or coding sequences (prokaryotic, and eukaryotic) can be inserted into an adenovirus vector, in accordance with the present invention, particularly to provide protection against a wide range of diseases.

A heterologous (i.e., foreign) nucleotide sequence or transgene may comprise one or more gene(s) of interest, and may have therapeutic or diagnostic value. In the context of the present invention, a gene of interest can code either for an antisense RNA, a ribozyme or for an mRNA which will then be translated into a protein of interest. A gene of interest can be of genomic type, of complementary DNA (cDNA) type or of mixed type (minigene, in which at least one intron is deleted). It can code for a mature protein, a precursor of a mature protein, in particular a precursor intended to be secreted and accordingly comprising a signal peptide, a chimeric protein originating from the fusion of sequences of diverse origins, or a mutant of a natural protein displaying improved or modified biological properties. Such a mutant can be obtained by deletion, substitution and/or addition of one or more nucleotide(s) of the gene coding for the natural protein, or any other type of change in the sequence encoding the natural protein, such as, for example, transposition or inversion.

In some cases the gene for a particular antigen can contain a large number of introns or can be from an RNA virus, in these cases a complementary DNA copy (cDNA) can be used. It is also possible that only fragments of nucleotide sequences of genes can be used (where these are sufficient to generate a protective immune response or a specific biological effect) rather than the complete sequence as found in the wild-type organism. Where available, synthetic genes or fragments thereof can also be used. However, the present invention can be used with a wide variety of genes, fragments and the like, and is not limited to those set out above.

Recombinant vectors of the present invention can be used to express antigens for provision of, for example, subunit vaccines. Antigens used in the present invention can be either native or recombinant antigenic polypeptides or fragments. They can be partial sequences, full-length sequences, or even fusions (e.g., having appropriate leader sequences for the recombinant host, or with an additional antigen sequence for another pathogen). An antigenic polypeptide to be expressed by the virus systems of the present invention may contain full-length (or near full-length) sequences encoding antigens or shorter sequences that are antigenic (i.e., encode one or more epitopes). The shorter sequence can encode a “neutralizing epitope,” which is defined as an epitope capable of eliciting antibodies that neutralize virus infectivity in an in vitro assay. Preferably the peptide should encode a “protective epitope” that is capable of raising in the host a “protective immune response;” i.e., a humoral (i.e. antibody-mediated), cell-mediated, and/or mucosal immune response that protects an immunized host from infection.

The antigens used in the present invention, particularly when comprised of short oligopeptides, can be conjugated to a vaccine carrier. Vaccine carriers are well known in the art: for example, bovine serum albumin (BSA), human serum albumin (HSA) and keyhole limpet hemocyanin (KLH). A preferred carrier protein, rotavirus VP6, is disclosed in EPO Pub. No. 0259149, the disclosure of which is incorporated by reference herein.

Genes for desired antigens or coding sequences thereof which can be inserted include those of organisms which cause disease in mammals.

With the recombinant adenovirus vectors of the present invention, it is possible to elicit an immune response against disease antigens and/or provide protection against a wide variety of diseases affecting swine, cattle, humans and other mammals. Any of the recombinant antigenic determinants or recombinant live viruses of the invention can be formulated and used in substantially the same manner as described for the antigenic determinant vaccines or live vaccine vectors.

The present invention also includes compositions comprising a therapeutically effective amount of a recombinant adenovirus vector of the present invention, recombinant virus of the present invention or recombinant protein, prepared according to the methods of the invention, in combination with a pharmaceutically acceptable vehicle or carrier and/or an adjuvant. Such a composition can be prepared and dosages determined according to techniques that are well-known in the art. The pharmaceutical compositions of the invention can be administered by any known administration route including, but not limited to, systemically (for example, intravenously, intratracheally, intraperitoneally, intranasally, parenterally, enterically, intramuscularly, subcutaneously, intratumorally or intracranially) or by aerosolization or intrapulmonary instillation.

In some examples, the adenovirus or adenovirus vectors are particularly advantageous for use in oral vaccines of mammals. Administration can take place in a single dose or in doses repeated one or more times after certain time intervals. The appropriate administration route and dosage will vary in accordance with the situation (for example, the individual being treated, the disorder to be treated or the gene or polypeptide of interest), but can be determined by one of skill in the art.

Compositions and methods have been described for the oral immunization of humans and various animal species. For example, U.S. Pat. No. 5,352,448 discloses an oral vaccine formulation for ruminants, comprising an antigen composition in a delivery vehicle consisting of a water-swellable hydrogel matrix, which allows for delivery of the antigen to mucosa-associated lymphoid tissue in the post-ruminal portion of the digestive tract. U.S. Pat. No. 5,176,909 discloses compositions for oral administration to humans or animals, comprising an immunogen, gelatin of a particular molecular weight range, and an enteric coating. U.S. Pat. No. 5,075,109 discloses a method for targeting a bioactive agent, e.g., an antigen, to the Peyer's patches by microencapsulating the agent in a biocompatible polymer or copolymer, such as poly(DL-lactide-co-glycolide). U.S. Pat. No. 5,032,405 discloses an oral formulation, comprising a lyophilized mixture of a 1 5 biologically active agent, e.g., an immunogen, in combination with maltose, a particulate diluent, and a coating comprising an alkaline-soluble polymeric film. Additional references on encapsulation of adenovirus include Periwal et al., 1997, J. Virol. 71:2844; Bowersock et al., 1998, Immunology Letters, 60:37; and Mittal et al., 2000, Vaccine 19:253. U.S. Pat. No. 4,152,415 discloses a method of immunizing field-raised swine against dysentery, comprising administering a sequential series of parenteral and entericcoated oral preparations of a virulent isolate of killed cells of Treponema hyodysenteriae.

The vaccines of the invention carrying foreign genes or fragments can be orally administered in a suitable oral carrier, such as in an enteric-coated dosage form. Oral formulations include such normally-employed excipients as, for example, pharmaceutical grades of mannitol, lactose, starch, magnesium stearate, sodium saccharin cellulose, magnesium carbonate, and the like. Oral vaccine compositions may be taken in the form of solutions, suspensions, tablets, pills, capsules, sustained release formulations, or powders, containing from about 10% to about 95% of the active ingredient, preferably about 25% to about 70%. An oral vaccine may be preferable to raise mucosal immunity (which plays an important role in protection against pathogens infecting the gastrointestinal tract) in combination with systemic immunity. U.S. Pat. No. 6,387,397 discloses polymerized liposomes for oral and/or mucosal delivery of vaccines.

Protocols for administering to individuals the vaccine composition(s) of the present invention are within the skill of the art in view of the present disclosure. Those skilled in the art will select a concentration of the vaccine composition in a dose effective to elicit antibody, cell-mediated and/or mucosal immune responses to the antigenic fragment. Within wide limits, the dosage is not believed to be critical. Typically, the vaccine composition is administered in a manner which will deliver between about 1 to about 1,000 micrograms of the subunit antigen in a convenient volume of vehicle, e.g., about 1-10 ml. Preferably, the dosage in a single immunization will deliver from about 1 to about 500 micrograms of subunit antigen, more preferably about 5-10 to about 100-200 micrograms (e.g., 5-200 micrograms).

The timing of administration may also be important. For example, a primary inoculation preferably may be followed by subsequent booster inoculations, for example, several weeks to several months after the initial immunization, if needed. To insure sustained high levels of protection against disease, it may be helpful to re-administer booster immunizations at regular intervals, for example once every several years. Alternatively, an initial dose may be administered orally followed by later inoculations, or vice versa. Preferred vaccination protocols can be established through routine vaccination protocol experiments.

The dosage for all routes of administration of in vivo recombinant virus vaccine depends on various factors including, the size of mammalian subject, nature of infection against which protection is needed, carrier and the like and can readily be determined by those of skill in the art. By way of non-limiting example, a dosage of between approximately 10³ pfu and 10⁸ pfu can be used. As with in vitro subunit vaccines, additional dosages can be given as determined by the clinical factors involved.

The invention also encompasses a method of treatment, according to which a therapeutically effective amount of an adenovirus vector, recombinant adenovirus, or host cell of the invention is administered to a mammalian subject requiring treatment.

When the heterologous sequences encode an antigenic polypeptide, adenovirus vectors comprising insertions of heterologous nucleotide sequences can be used to provide large quantities of antigen which are useful, in turn, for the preparation of antibodies. Methods for preparation of antibodies are well-known to those of skill in the art. Briefly, an animal (such as a rabbit) is given an initial subcutaneous injection of antigen plus Freund's complete adjuvant. One to two subsequent injections of antigen plus Freund's incomplete adjuvant are given at approximately 3 week intervals. Approximately 10 days after the final injection, serum is collected and tested for the presence of specific antibody by ELISA, Western Blot, immunoprecipitation, or any other immunological assay known to one of skill in the art.

Adenovirus E1 gene products transactivate many cellular genes; therefore, cell lines which constitutively express E1 proteins can express cellular polypeptides at a higher levels than other cell lines. The recombinant mammalian cell lines of the invention that comprise adenovirus encoding transgenes can be used to prepare and isolate polypeptides.

The invention also includes a method for delivering a gene to a mammal, such as a bovine, human or other mammal in need thereof, to control a gene deficiency. In one embodiment, the method comprises administering to said mammal a live recombinant adenovirus of the present invention containing a heterologous nucleotide sequence encoding a non-defective form of said gene under conditions wherein the recombinant virus vector genome is incorporated into said mammalian genome or is maintained independently and extrachromosomally to provide expression of the required gene in the target organ or tissue. These kinds of techniques are currently being used by those of skill in the art to replace a defective gene or portion thereof. Examples of foreign genes, such as transgenes, heterologous nucleotide sequences, or portions thereof that can be incorporated for use in gene therapy include, but are not limited to, growth factors, cytokines and the like.

The present invention is not to be limited in scope by the specific embodiments described herein. Various modifications of the invention in addition to those described herein will become apparent to those skilled in the art from the foregoing description and accompanying figures. Such modifications are intended to fall within the scope of the appended claims.

### EXAMPLE 1

Construction of the Recombinant Plasmids Containing the Modified pIX Gene of BAV-3

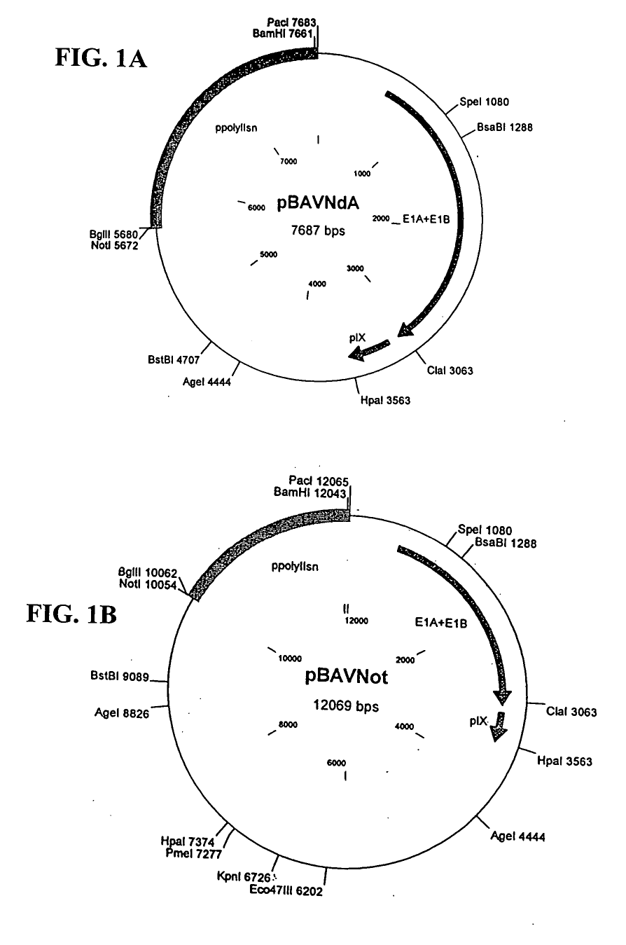

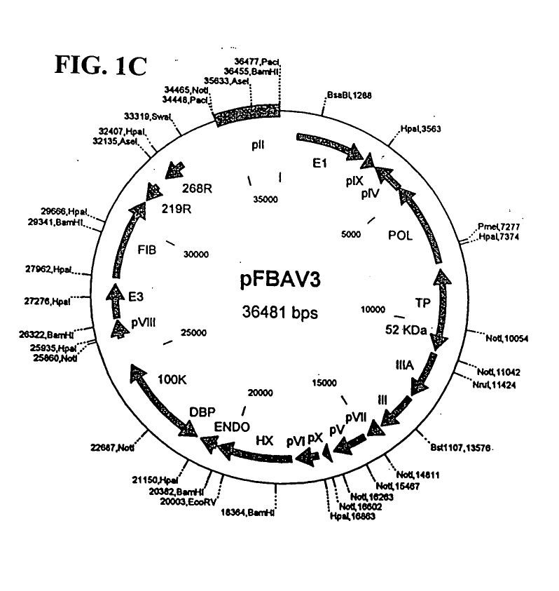

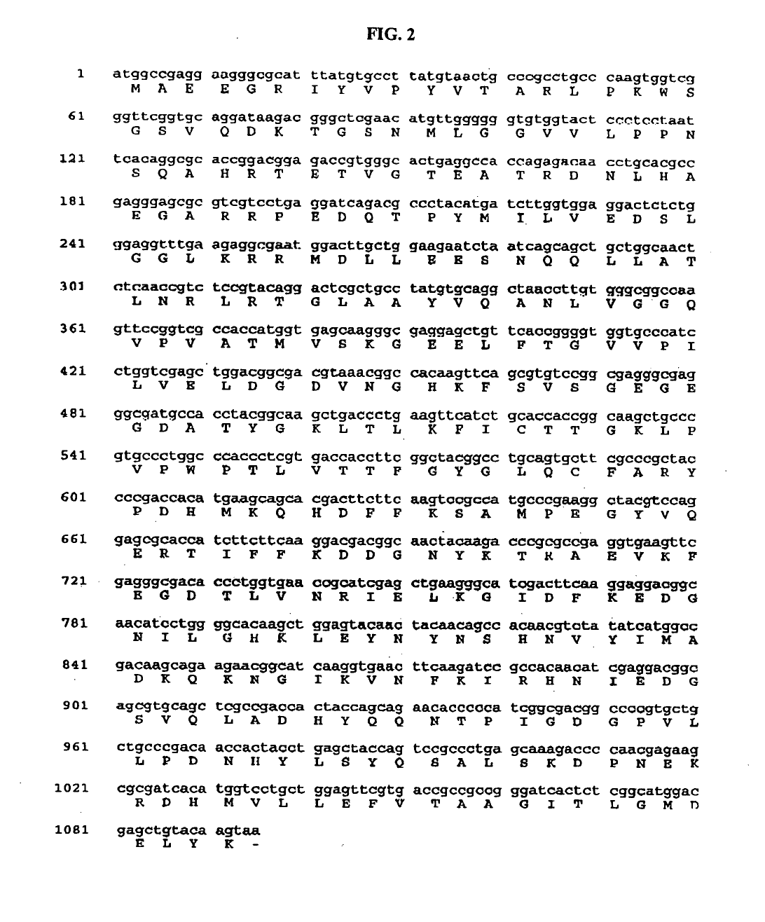

The gene for enhanced yellow fluorescent protein (EYFP) was obtained from pEYFP—N1 (CLONTECH) by digesting the DNA with Agel and NotI. The 731 bp fragment was blunted with Klenow and cloned into HpaI site pBAVNdA (FIG. 1A).

The overlapping synthetic oligonucleotides were used to make DNA sequence containing the RGD motif. Sense oligo sequence is:

GGATCAGGATCAGGTTCAGGGAGTGGCTCTCGCCTGCGACTGTCGCGG
CGATTGTTTTTGCGGTTAAGTT

and antisense is:

AACTTAACCGCAAAAACAATCGCCGCGACAGTCGCAGGCAGAGCCACT
CCCTGAACCTGATCCTGATCC.

The oligonucleotides were mixed together and cloned into the HpaI site of pBAVNdA (FIG. 1A). The resulting plasmids were named pBNdAYFP and pBNdARGD respectively.

The 4382 by Agel fragment of the BAV-3 genome was inserted into Agel site of pBNdAYFP and pBNdARGD to extend homologous sequences for the following recombination in E. coli. The resulting plasmids were named pBAVNotYFP and pBAVNotRGD (see FIG. 1B). pBAVNotYFP and pBAVNotRGD were cut by PacI and NotI, and the larger fragment was used for the homologous recombination with pFBAV3 DNA digested with BsaBI and PmeI (FIG. 1C). The recombination was carried out in the E. coli strain BJ5183. The resulting full-length genomic plasmids were named pFBAV951 (EYFP) and pFBAV950 (RGD).

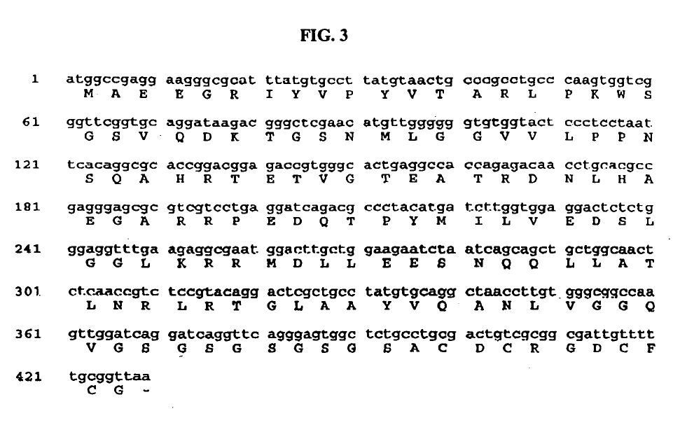

The sequence of the chimerical pIX gene fused with EYFP is in FIG. 2, and the sequence of the chimerical pIX gene fused with the RGD-containing peptide is in FIG. 3.

### EXAMPLE 2

Construction of the Recombinant Bav-3 Viruses with Modified pIX Gene

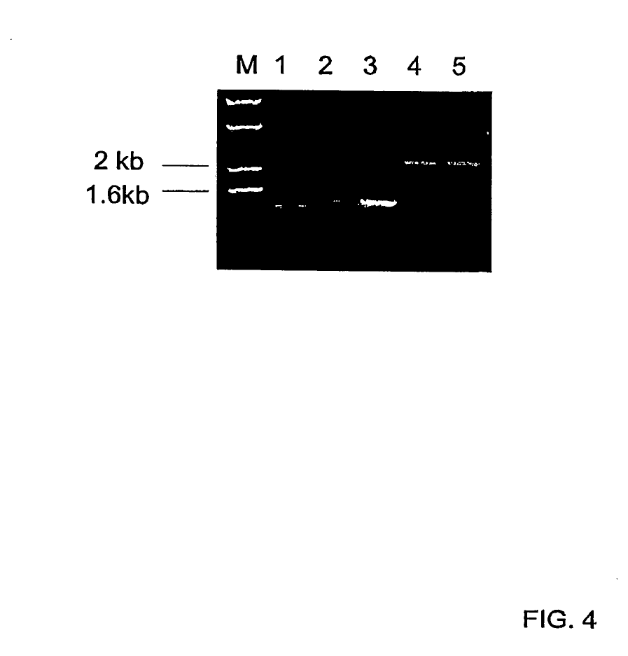

5 μg of DNA of pFBAV951 and pFBAV950 was digested with PacI and used for transfection of VIDO-R2 cells by a Lipofectin method. The viral plaques appeared 14 days after transfection. The insertion of the foreign sequences was analyzed by PCR on the viral DNA. The primers used for analysis: P91 CTAATCGATACATGTACACTG (3057 bp of BAV-3 genome) and P92 CCAACCGGTTGTGGAAAATC (4450 bp of BAV-3 genome). The PCR product, generated on wild-type genome, was 1393 base pairs (bp) in length (FIG. 4; lane 1). In the result of the insertion of the RGD-containing sequence, the product length has increased to 1456 bp (FIG. 4; lane 2). In the result of the insertion of the EYFP sequence, the product length increased to 2125 bp (FIG. 4; lane 4). There was no difference between the length of the products from the viral DNA passage 2 and 10 (FIG. 4, lane 2 and 3; 4 and 5). This provides evidence that genomes of the recombinants are stable.

### EXAMPLE 3

Incorporation of Modified pIX into the Viral Capsid

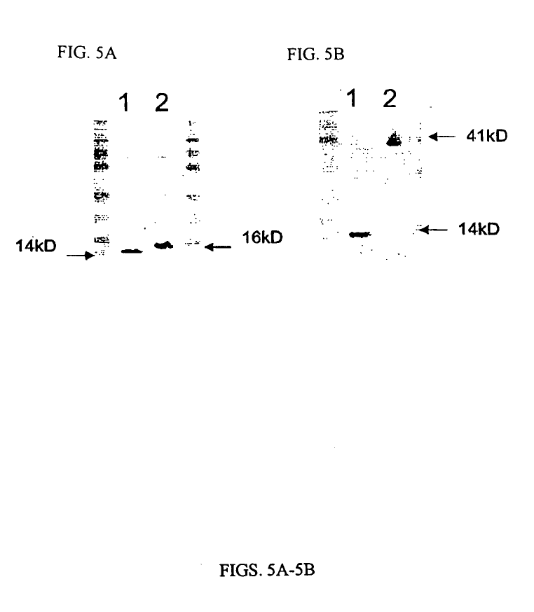

The recombinants and wild-type BAV-3 were purified by ultracentrifugation in a gradient of CsCl. The proteins of purified virions were separated on 12% denaturing PAAG and analyzed in Western blotting using rabbit polyclonal anti-sera against BAV-3 pIX. Anti-pIX sera recognized a protein of 14 kDa in case of wild-type virus, 16 kDa protein in case of the RGD containing recombinant BAV950 and 41 kDa protein in case of the EYFP containing recombinant BAV951 (FIGS. 5A-5B).

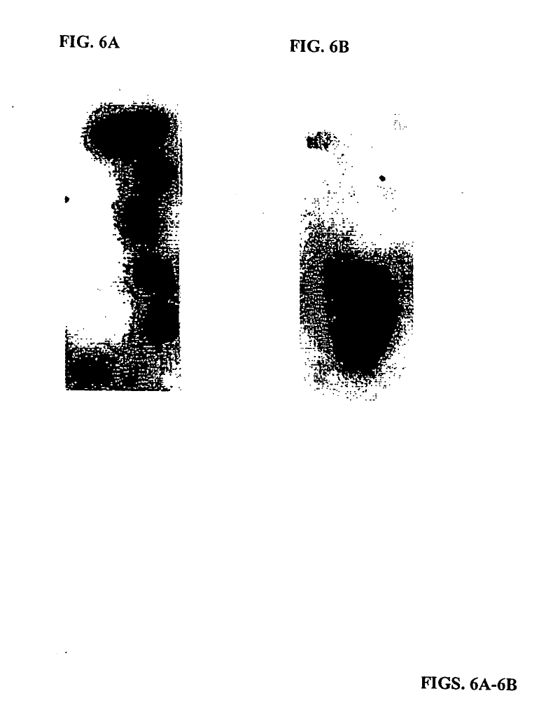

To prove the surface location of EYFP in the virion, immunoelectron microscopy was performed with purified virions and anti-EYFP sera. For immunogold electron microscopy, purified virions were adsorbed to nickel grids. After adsorption, the grids were incubated for 1 hr with appropriately diluted anti-sera. After several washing steps, the grids were incubated with gold-tagged protein A. for 1 hr at room temperature. The grids were stained with 2% phosphotungstic acid and examined by transmission electron microscopy. As can be seen in FIGS. 6A-6B, the BAV951 virions were labeled with EYFP-specific antibodies and gold-tagged protein A, whereas the BAV-3 virions did not react with anti-EYFP sera.

In summary, these data demonstrate incorporation of chimerical pIX into the viral particles and the external localization of the EYFP protein, fused with pIX.

### EXAMPLE 4

Infection Efficiency of pIX Modified BAV-3

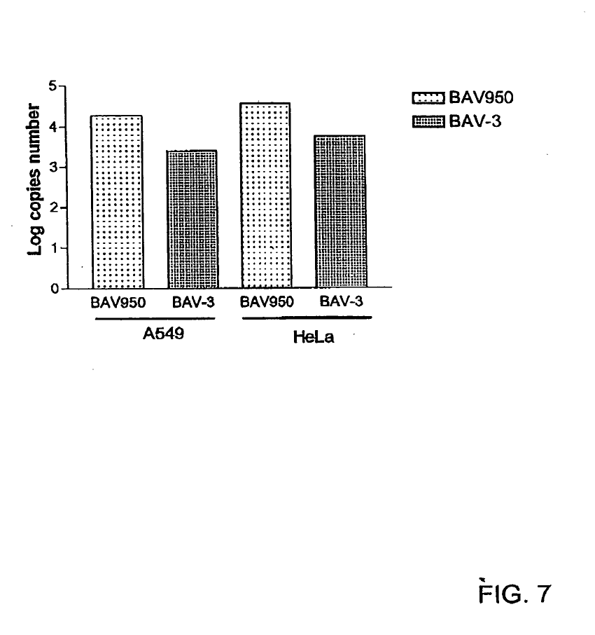

To evaluate whether the incorporation of RGD into pIX would improve the efficiency of infection, the integrin-containing cells (HeLa and A549) were infected with BAV-3 or BAV950 at multiplicity of infection (m.o.i.) 100 TCID₅₀/cell. After 2 hr of adsorption, cells were washed twice with PBS and media was changed to MEM+10% FBS. At 48 hr after infection, cells were trypsinized and harvested. Total DNA was extracted from the cells, using QIAGEN DNAeasy Tissue Kit. An Aliquot of 70 ng of total DNA was used in the Real Time PCR analysis. The primers were from the BAV-3 hexon gene sequence: RTP-1 TACAGTAATGTGGCGTTGTA and RTP-2 CGTATCAATAAGGCCGCTAA. The 5′-end labeled FAM (6-carboxy-fluorescein, reporter dye) and 3′-labeled TAMRA (6-carboxytetramethyl-fhodamine, quencher dye) probe was used in the PCR reaction. The sequence of the probe is CCGCCTAACCACGAACACCTACG. Dilutions of pFBAV3 DNA were used for absolute quantification of viral genomes in the DNA sample. As can be seen in FIG. 7, 10 fold more viral DNA was found in BAV950 infected cells, as compared to the BAV-3 infected cells, for both cell lines.

### EXAMPLE 5

Production of Antibody to Cells of Small Intestine Materials and Methods:

BALB/c mice (10-12 weeks old) were immunized intraperitoneally (I/P) with 100 μg of membranous antigen in Complete Freund's Adjuvant (CFA) obtained from sheep jejunal Peyer's patch (JPP) epithelial cells. Mice were boosted 21, 35, and 45 days after first immunization with the same amount of Ag in Incomplete Freud's adjuvant (IFA). One immunized mouse was killed 5 days after the last boost and spleen cells were fused with NS-1 myeloma cells. The supernatants obtained from the hybridomas were tested on JPP epithelial cells by FACS in order to select hybridomas secreting MoAbs specific for the cell surface molecules. Epithelial cells for FACS analysis were obtained from sheep JPPs by EDTA or collagenase digestion. In total 181 clones were obtained and 36 clones were found positive for JPP epithelial cells by FACS analysis. Some of clones positive by FACS were tested on JPP tissues by immunohistochemical (ICH) staining. Four clones positive both by FACS and IHC for epithelial cell staining were further sub cloned by limiting dilution method. Isotyping of MoAbs was done using JPP epithelial cells staining and various mouse Ab isotype specific FITC conjugates. All the four MoAbs were found to be of IgM isotype. These four MoAb supernatants were further characterized by FACS using sheep jejunal PP epithelial cells and by ICH staining using sheep JPP, ileal PP (IPP) and jejunum tissues. Cross-reactivity of these MoAbs with other species was tested by IHC staining. These MoAbs cross-reacted with both bovine and porcine JPP, IPP and jejunum tissues.

Numerous infectious agents enter the body through the mucosal surfaces of the gastrointestinal tract. A prerequisite for inducing mucosal immune responses in the intestinal tract, is the efficient transepithelial transport of antigens to gut-associated lymphoid tissues (GALT). Specialized epithelial M cells, localized in the follicle-associated epithelium (FAE) of the Peyer's patches (PP), efficiently deliver foreign antigens to GALT. Antibodies identified by the method described above are used in the production of adenovirus vectors comprising chimeric capsid proteins. Such adenovirus vectors are used in vaccine compositions and methods for the delivery of antigens to mammalian GALT tissue. The identification of antibodies that cross-react with bovine, ovine and porcine cells present in GALT are used in the preparation of adenovirus vectors that can be used in vaccine protocols for multiple mammalian species.

1. A chimeric adenovirus capsid protein wherein said protein comprises a part of or all of an adenovirus capsid protein and a binding partner of a cell-surface binding site present in a cell of gut associated lymphoid tissue (GALT) of a mammal, wherein said chimeric adenovirus capsid protein is capable of binding to the cell.

2. The chimeric adenovirus capsid protein of claim 1 wherein said capsid protein is selected from the group consisting of hexon, penton, fiber pIX, and IIIa.

3. The chimeric adenovirus capsid protein of claim 1 wherein said adenovirus is mammalian adenovirus.

4. The chimeric adenovirus capsid protein of claim 3 wherein said adenovirus is a ruminant mammalian adenovirus.

5. The chimeric adenovirus capsid protein of claim 3 wherein said mammalian adenovirus is selected from the group consisting of a human, porcine, bovine, and ovine.

6. The chimeric adenovirus capsid protein of claim 1 wherein said adenovirus capsid protein is protein IX (pIX).

7. The chimeric adenovirus capsid protein of claim 1 wherein said adenovirus capsid protein is a fiber protein.

8. The chimeric adenovirus capsid protein of claim 2 wherein said binding partner is an antibody, or a fragment thereof.

9. The chimeric adenovirus capsid protein of claim 8 wherein said antibody binds an epithelial cell present in the GALT.

10. The chimeric adenovirus capsid protein of claim 8 wherein said antibody binds a cell present in mammalian Peyer's patches.

11. The chimeric adenovirus capsid protein of claim 8 wherein said antibody binds a microfold (M) cell.

12. The chimeric adenovirus capsid protein of claim 8 wherein said antibody binds a protein present on the surface of the cell.

13. The chimeric adenovirus capsid protein of claim 8 wherein said antibody binds a carbohydrate present on the surface of the cell.

14. The chimeric adenovirus capsid protein of claim 1 wherein said protein is encoded by a polynucleotide comprising nucleic acid encoding a part of or all of said capsid protein and nucleic acid encoding an amino acid sequence for said binding partner.

15. The chimeric adenovirus capsid protein of claim 1 wherein said protein comprises part or all of the capsid protein conjugated to the binding partner.

16. An adenovirus capsid comprising a chimeric adenovirus capsid protein.

17. A complex comprising a chimeric adenovirus capsid protein bound to a cell-surface binding site present in a cell of GALT of a mammal.

18. A recombinant vector comprising a polynucleotide encoding the chimeric adenovirus capsid protein of claim 1.

19. The vector of claim 18 wherein said vector is an adenovirus vector.

20. The vector of claim 19 wherein said adenovirus vector is a mammalian adenovirus vector.

21. The vector of claim 20 wherein said mammalian adenovirus is selected from the group consisting of human, porcine, bovine and sheep adenovirus.

22. The vector of claim 20 wherein said mammalian adenovirus vector is a ruminant adenovirus vector.

23. The vector of claim 18 wherein said vector comprises adenovirus sequences essential for encapsidation.

24. The vector of claim 23 wherein said adenovirus sequences essential for encapsidation are bovine adenovirus sequences.

25. The vector of claim 23 wherein said adenovirus sequences essential for encapsidation are porcine adenovirus sequences.

26. The vector of claim 19 wherein said adenovirus is a replication-competent adenovirus vector.

27. The vector of claim 26 wherein said replication-competent adenovirus further comprises a polynucleotide encoding a heterologous protein.

28. The vector of claim 19 wherein said adenovirus vector is replication-deficient.

29. The vector of claim 28 wherein said adenovirus vector is a replication-deficient bovine adenovirus vector.

30. The vector of claim 29 wherein said replication-deficient bovine adenovirus vector lacks E1 function.

31. The vector of claim 30 wherein said bovine adenovirus vector comprises a deletion of part or all of the E1 gene region.

32. The vector of claim 31 further comprising a deletion of part or all of the E3 gene region.

33. The vector of claim 29 wherein said vector further comprises a polynucleotide encoding a heterologous protein.

34. The vector of claim 33 wherein said heterlogous protein is an antigen of a pathogen.

35. A host cell comprising a vector of claim 18.

36. A viral particle comprising a vector of claim 18.

37. A composition comprising a vector of claim 18.

38. The composition of claim 37 further comprising a pharmaceutically acceptable excipient.

39. A vaccine composition comprising a vector of claim 18.

40. An immunogenic composition comprising a vector of claim 18, and a pharmaceutically acceptable excipient.

41. A method for eliciting an immune response in a mammalian host comprising administering the immunogenic composition of claim 40 to said mammalian host.

42. The method of claim 41 wherein said immunogenic composition is administered orally.

43. The method of claim 42 wherein said mammalian host is a ruminant mammal.

44. The method of claim 43 wherein said ruminant mammal is a bovine or ovine mammal.

45. A method for producing a chimeric adenovirus capsid protein comprising conjugating a binding partner of a cell-surface binding site of a cell present in the GALT of a mammal to part of or all of an adenovirus capsid protein wherein said capsid protein is located on the surface of the adenovirus capsid.

46. The method of claim 45 wherein the adenovirus capsid protein is selected from the group consisting of hexon, penton, fiber, pIX and IIIa.

47. A method for preparing a recombinant adenovirus vector comprising a polynucleotide encoding a chimeric adenovirus capsid protein comprising the steps of culturing a suitable host cell transformed with an adenovirus vector of claim 18 under conditions suitable to allow formation of a virus particle from said vector and optionally recovering the virus.


SEQUENCE LISTING (25 sequences)

SEQ ID NO: 1 - DNA, 16 bp, Porcine Adenovirus
cggaaattcccgcaca

SEQ ID NO: 2 - DNA, 17 bp, Porcine Adenovirus
gggattttgtgccctct

SEQ ID NO: 3 - DNA, 16 bp, Porcine Adenovirus
cggtattccccacctg

SEQ ID NO: 4 - DNA, 18 bp, Porcine Adenovirus
gtgtattttttcccctca

SEQ ID NO: 5 - DNA, 17 bp, Porcine Adenovirus
gtgtatatagtccgcgc

SEQ ID NO: 6 - DNA, 16 bp, Porcine Adenovirus
gagttttctctcagcg

SEQ ID NO: 7 - DNA, 18 bp, Porcine Adenovirus
ggcggaaattcccgcaca

SEQ ID NO: 8 - DNA, 19 bp, Porcine Adenovirus
gcgggattttgtgccctct

SEQ ID NO: 9 - DNA, 18 bp, Porcine Adenovirus
cccggtattccccacctg

SEQ ID NO: 10 - DNA, 20 bp, Porcine Adenovirus
gggtgtattttttcccctca

SEQ ID NO: 11 - DNA, 19 bp, Porcine Adenovirus
cagtgtatatagtccgcgc

SEQ ID NO: 12 - DNA, 18 bp, Porcine Adenovirus
tagagttttctctcagcg

SEQ ID NO: 13 - DNA, 14 bp, Porcine Adenovirus
ctggtattttccac

SEQ ID NO: 14 - DNA, 10 bp, Porcine Adenovirus
gtgatattgg

SEQ ID NO: 15 - DNA, 12 bp, Porcine Adenovirus
cctttacctggg

SEQ ID NO: 16 - DNA, 14 bp, Porcine Adenovirus
ctcaattttaccac

SEQ ID NO: 17 - DNA, 15 bp, Porcine Adenovirus
ggtcgatttttccac

SEQ ID NO: 18 - DNA, 18 bp, Porcine Adenovirus
ccctatttattctgcgcg

SEQ ID NO: 19 - DNA, 70 bp, Artificial Sequence
  Primer
ggatcaggatcaggttcagggagtggctctcgcctgcgactgtcgcggcgattgtttttgcggttaagtt

SEQ ID NO: 20 - DNA, 69 bp, Artificial Sequence
  Primer
aacttaaccgcaaaaacaatcgccgcgacagtcgcaggcagagccactccctgaacctgatcctgatcc

SEQ ID NO: 21 - DNA, 21 bp, Artificial Sequence
  Primer
ctaatcgatacatgtacactg

SEQ ID NO: 22 - DNA, 20 bp, Artificial Sequence
  Primer
ccaaccggttgtggaaaatc

SEQ ID NO: 23 - DNA, 20 bp, Artificial Sequence
  Primer
tacagtaatgtggcgttgta

SEQ ID NO: 24 - DNA, 20 bp, Artificial Sequence
  Primer
cgtatcaataaggccgctaa

SEQ ID NO: 25 - DNA, 23 bp, Artificial Sequence
  Primer
ccgcctaaccacgaacacctacg